(19) United States
(12) Patent Application Publication
(10) Pub. No.: US 2025/0127826 A1
(43) Pub. Date: Apr. 24, 2025

(54) Engineered Probiotics for Colorectal Cancer Screening, Prevention, and Treatment

(71) Applicant: The Trustees of Columbia University in the City of New York, New York, NY (US)

(72) Inventors: Tal DANINO, Brooklyn, NY (US); Candice GURBATRI, Dix Hills, NY (US)

(73) Assignee: The Trustees of Columbia University in the City of New York, New York, NY (US)

(21) Appl. No.: 18/936,495

(22) Filed: Nov. 4, 2024

Related U.S. Application Data
(63) Continuation-in-part of application No. PCT/US2023/066585, filed on May 4, 2023.
(60) Provisional application No. 63/557,150, filed on Feb. 23, 2024.
(60) Provisional application No. 63/338,132, filed on May 4, 2022.

(51) Int. Cl. A61K 35/741; A61K 35/00; A61P 35/00; C07K 14/535; C07K 16/28; C12N 15/70; G01N 33/53; G01N 33/574

(52) U.S. Cl. CPC A61K 35/741; A61P 35/00; C07K 14/535; C07K 16/2818; C07K 16/2827; C12N 15/70; G01N 33/5308; G01N 33/57419; A61K 2035/115; C07K 2317/569; C12N 2800/101

(57) ABSTRACT

Orally-deliverable programmable bacteria cells that colonize colorectal tumors and produce diagnostic and therapeutic molecules, as well as related compositions and methods.

### CROSS-REFERENCE TO RELATED APPLICATIONS

The instant application is a bypass continuation-in-part application of International Patent Application No. PCT/US23/066585 filed 4 May 2023 and claims priority to U.S. Provisional Patent Applications Nos. 63/338,132, filed 4 May 2022, and 63/557,150, filed 23 Feb. 2024, which is hereby incorporated by reference in its entirety.

### STATEMENT REGARDING FEDERALLY SPONSORED RESEARCH OR DEVELOPMENT

This invention was made with government support under grants U01CA247573 and R01CA249160 awarded by the National Institutes of Health and 1644869 awarded by the National Science Foundation. The government has certain rights in the invention.

### SEQUENCE LISTING

The instant application contains a Sequence Listing which has been submitted in XML format via EFS-Web and is hereby incorporated by reference in its entirety. Said XML copy, created on 3 May 2023, is named 39700051WO01SEQL.xml and is 7,245 bytes in size.

### TECHNICAL FIELD OF THE INVENTION

This disclosure generally relates to the fields of medicine and immunology. More specifically, the disclosure relates to orally-deliverable programmable bacteria cells (e.g., E. coli Nissle 1917 bacteria) that colonize colorectal tumors and produce diagnostic and therapeutic molecules, as well as related compositions and methods.

### BACKGROUND OF THE INVENTION

Colorectal cancer (CRC) is the second leading cause of morbidity and mortality worldwide, with incidence rates significantly rising in younger populations. Colonoscopies are effective at reducing CRC incidence and mortality, allowing for simultaneous detection and treatment with removal of precancerous and cancerous lesions, however they are (1) inconvenient, as they require bowel preparation and a day off work; (2) costly due to hospital and personnel expenses; and (3) carry some risk, both procedural and related to anesthesia. Given this, CRC screening often occurs as a two-step approach, firstly a non-invasive step such as the immunochemical fecal occult blood test (iFOBT), which if positive is then investigated by a diagnostic colonoscopy. Non-invasive stool-based tests that exist presently have variable sensitivity levels (˜5-40%) for pre-cancerous polyps, which are the primary precursor lesions of CRC and the target for CRC prevention. Beyond these limitations, patient adherence remains low for stool-based tests and is the main challenge to screening implementation. In contrast, serum-based tests are patient-preferred, but have lower sensitivity and specificity than the FIT tests.

None of the foregoing alternatives offer the opportunity to manipulate lesions in situ, resulting in the eventual need for therapeutic intervention. Colonoscopies and colectomies are therapeutic options to remove local disease. If the cancer become metastatic, chemotherapy is often administered and, in some cases, the lesion is able to be surgically resected. More recently, targeted immunotherapies have become available to manage disease, but patient response is variable, and microsatellite stable CRC (MSS-CRC) is not responsive to immunotherapy in most cases due to the lack of immune cell infiltration.

The ideal CRC prevention strategy would address current issues of cost, compliance, complication, and provide direct and effective chemo-, immune-, or bio-prevention to prevent progression from benign to malignant disease.

### BRIEF SUMMARY OF THE INVENTION

The present disclosure relates programmable bacterial cells for diagnosing and/or treating colorectal tumors in a subject (e.g., a human). In some embodiments, programmable bacterial cells described herein comprise (i) a first nucleic acid encoding an isochorismate synthase; and (ii) a second nucleic acid encoding an isochorismate-pyruvate lyase. In other embodiments, the programmable bacterial cells described herein comprise a knockout of one or more genes encoding a lactate dehydrogenase, a phosphate acetyltransferase, an acetaldehyde-CoA dehydrogenase, and a fumarate reductase, and further comprises: (i) a first nucleic acid encoding an acetyl-CoA acetyltransferase; (ii) a second nucleic acid encoding a 3-hydroxybutyryl-CoA dehydrogenase; (iii) a third nucleic acid encoding a crotonase; (iv) a fourth nucleic acid encoding a trans-enoyl-CoA reductase; and (v) a fifth nucleic acid encoding an acyl-CoA thioesterase.

When administered to a subject with one or more colorectal tumors, the programmable bacterial cells multiply within the colorectal tumor microenvironment (TME). Over time, the programmable bacterial cells produce an analyte (salicylate or butyrate), which is detectable in a biological sample obtained from the subject, such as stool or urine. Levels of the analyte in the biological sample may then be compared against reference values, and a conclusion can then be drawn as to whether or not the subject has one or more colorectal tumors.

In some embodiments, the programmable bacterial cells belong to at least one genus selected from the group consisting of Salmonella, Escherichia, Firmicutes, Bacteroidetes, Lactobacillus, and Bifidobacteria. In some embodiments, the programmable bacterial cells belong to the genus Escherichia. In particular embodiments, the programmable bacterial cells are Escherichia coli Nissle (EcN) cells. In some embodiments, the EcN cells comprise a knockout of the clbA gene (EcNΔclbA). In some embodiments, the EcN cells comprise genomically integrated aroG, tkt and talB genes (EcN^{ATT}). In some embodiments, the programmable bacterial cells comprise a luxCDABE luciferase cassette (EcN-lux).

In some embodiments, the programmable bacterial cells comprise a synchronized lysis circuit comprising a nucleic acid encoding a quorum-sensing gene, a nucleic acid encoding a lysis gene, a promoter, and a terminator contained on a single operon and one or more nucleic acids encoding a diagnostic agent for diagnosing colorectal tumors and/or one or more nucleic acids encoding a therapeutic agent for treating colorectal tumors.

In some embodiments, the programmable bacterial cells comprise a therapeutic agent. In these embodiments, the programmable bacteria cell further comprises one or more nucleic acids encoding (a) an antibody that specifically binds to PD-L1; (b) an antibody that specifically binds to CTLA-4; (c) a cytokine; (d) an antibody that specifically binds to PD-L1 and an antibody that specifically binds to CTLA-4; (e) an antibody that specifically binds to PD-L1 and a cytokine; (f) an antibody that specifically binds to CTLA-4 and a cytokine; and (g) an antibody that specifically binds to PD-L1, an antibody that specifically binds to CTLA-4, and a cytokine (e.g., GM-CSF).

The present disclosure also relates to methods of monitoring the treatment of a colorectal tumor in a subject comprising administering a programmable bacterial cell described herein to the subject, wherein the programmable bacterial cell comprises a nucleic acid encoding an analyte described herein, which is detectable in a biological sample obtained from the subject; obtaining a first biological sample from the subject at a first time point; measuring the level of the analyte in the first biological sample; obtaining a second biological sample from the subject at a second time point; and measuring the level of the same analyte in the second biological sample. Clinically significant differences in levels of the analyte in the first and second samples are indicative of the presence of one or more colorectal tumors in the subject.

The present disclosure also relates to methods of treating a colorectal tumor in a subject comprising administering a therapeutically effective amount of programmable bacterial cells described herein to the subject, wherein the programmable bacterial cells comprise a nucleic acid encoding a therapeutic agent described herein, which are capable of treating the colorectal tumor.

The present disclosure also relates to methods of reducing the rate of proliferation of a colorectal tumor comprising delivering a programmable bacterial cell described herein to the colorectal tumor. The present disclosure also relates to methods of killing a colorectal tumor cell comprising delivering a programmable bacterial cell described herein to the colorectal tumor cell.

In some embodiments, the programmable bacterial cells described herein may be administered to a subject or delivered to a tumor in the form of a pharmaceutical composition, which may comprise one or more pharmaceutically acceptable carriers, diluents, or excipients.

The present disclosure also relates to articles of manufacture useful for treating a colorectal tumor. In some embodiments, the articles of manufacture comprise a container comprising programmable bacterial cells described herein, or pharmaceutical compositions comprising the same, as well as instructional materials for using the same to treat a colorectal tumor. In some embodiments, the articles of manufacture are part of a kit that comprises a bacterial culture vessel and/or bacterial cell growth media.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description of the Invention. This summary is not intended to identify key features or essential features of the claimed subject matter, nor is it intended to be used as an aid in determining the scope of the claimed subject matter.

### BRIEF DESCRIPTION OF THE SEVERAL VIEWS OF THE DRAWINGS

FIGS. 1A-1I show adenoma colonization of E. coli Nissle 1917 (EcN) in a genetically-engineered mouse model of CRC. (A) Schematic of spontaneous intestinal adenomas in Apc^{Min/+} model. 12-week-old Apc^{Min/+} mice were gavaged twice, 3-4 days apart with EcN expressing luxCDABE luciferase cassette (EcN-lux). (B) EcN-lux was visualized using an IVIS for bioluminescence in vivo 96 h post dosing. After 7 weeks, mice were sacrificed, intestinal tissue was excised and imaged ex vivo for bioluminescence. Red arrows point to macroadenomas on distal intestinal tissue (n=20 WT, n=25 Apc^{Min/+}). (C) Plot where x-axis is bioluminescence signal measured from ex vivo images of dissected intestinal tissue and y-axis is total adenoma area in matched tissue sections as measured in H&E-stained images (r=0.75, Spearman correlation coefficient). (D-E) In separate cohorts, mice were dosed with EcN-lux or EcN producing an HA-tagged protein and (D) sacrificed at 1 week post dosing. Intestinal tissue was homogenized and plated on antibiotic-selective plates for EcN-lux to quantify colony-forming-units (CFU) per gram of tissue (n=5 WT, n=5 Apc^{Min/+}, **** p<0.0001) or (E) sacrificed at 4 weeks post dosing and intestinal tissue was paraffin-embedded, sectioned and stained by anti-HA immunohistochemistry. Dark brown stain depicts HA-tagged protein produced by EcN in adenomas. Scale bars represent 200 μm (top) and 50 μm (bottom). (F) Schematic (Top) of colibactin-encoding operon in EcN whereby clbA is knocked out and colibactin production is disrupted. Plate reader experiment of strain variant growth kinetics at 0.1 and 0.01 seeding OD. (G-I) 12-week-old Apc^{Min/+} mice and WT littermates were gavaged twice, 3-4 days apart with 10 CFU bioluminescent EcN-lux or EcNΔclbA-lux. (G) After 1 week, mice were sacrificed, intestinal tissue was excised and ex vivo imaged for bioluminescence. Red arrows point to macroadenomas on distal small intestinal tissue (representative image from n=5 mice). (H) Body weight of both EcN-lux and EcNΔclbA-lux-treated mice were tracked over the course of the experiment (n=3-4 mice per group, 2-way ANOVA, ns, not significant). (I) In a separate cohort one week post dosing, mice were sacrificed, small intestine (SI), colon (Co), cecum (Ce), liver (L), and spleen (S) were harvested, homogenized, and plated on antibiotic selective plates to recover EcNΔclbA-lux per gram of tissue (n=3-6 mice per group). All error bars represent S.E.M.

FIGS. 2A-2K show tumor colonization of E. coli Nissle 1917 (EcN) in orthotopic mouse models and human CRC patients. (A) Schematic of experimental timeline for MSS CRC model. Tumor growth was monitored by colonoscopy, with animals orally dosed twice with EcN-lux or PBS before (B) Imaging 5 days after last dose for bioluminescence, with (C) luminescence quantified in organs ex vivo (L, liver, S, spleen, and NC, normal colon n=17, PT, proximal tumor, DT distal tumor, n=12, PBS tumors n=6). (D) Tissues were homogenized, plated on antibiotic-selective plates, and quantified for CFU per gram (n=17 from EcN-dosed non-tumor mice and EcN-dosed tumor mice n=6, PBS-dosed tumor mice n=4). (E & F) Representative images of orthotopic CRC from mice dosed as (A), with serial sections showing (E) EcN-lux specific location by RNASCOPE® in situ hybridization for lux (brown, scale bar 20 μm) and (F) immunofluorescent staining for HYPOXYPROBE® (red) and lipopolysaccharide (LPS, green, scale bar 10 μm, n=5 mice. (G) Schematic of experimental timeline for MSI CRC model. Tumor and non-tumor bearing control animals orally dosed thrice with EcN-lux before (H) Imaging 5 days after last dose with luminescence quantified in organs ex vivo and (I) tissues homogenized, plated on antibiotic-selective plates, and quantified for CFU per gram (n=4 for both tumor bearing and no-tumor control groups). (J) Schematic of human clinical trial. (K) Matched normal and tumor tissue homogenates from CRC patients administered placebo (n=2) or MUTAFLOR® (trade name for EcN) (n=6) for 2 weeks. Tissue samples were used to inoculate liquid culture for microbial enrichment. DNA isolated from culture was subjected to qPCR with an EcN specific assay. Mean value of four technical replicates shown per sample, box in the plot minima-maxima is 25th-75th percentile, whiskers at lowest and highest values, bar at median per group. Red dashed line depicts the lower limit of detection of each assay based on standard curve dilution series, dot points above the line have detectable PCR amplicon signal. No EcN signal was detected in E. coli control (ATCC Accession No. 9522), no template (NTC), or buffer only DNA prep controls. *p<0.05, Wilcoxon test. (C, D & I) **** p<0.0001, one-way ANOVA with Tukey's multiple comparisons. (H) **** p<0.0001, two-way ANOVA, Fischer's LSD test). (I) * p=0.03, two-way paired t-test.

FIGS. 3A-3G depict the engineering of stool and urine-based EcN platform for non-invasive adenoma tracking. (A) Schematic of orally-delivered EcN probiotic to be detected in fecal matter by quantifying colony-forming units or urine by metabolite quantification. (B) 15-week-old Apc^{Min/+} mice were dosed orally 3-4 days apart with EcN and one stool pellet was collected at 3, 7, 24, 48, and 72 h after last dose, homogenized, plated on antibiotic-selective plates, and quantified for CFU (n=5 mice per group). (C) Schematic of the genetic construct where the isochorismate synthase (ICS) and pchB genes result in salicylate production (Top). These enzymes were cloned onto plasmids and transformed into EcN or a metabolically engineered EcN strain (EcN^{ATT}). (D-E) Overnight cultures of these strain variants were optical density-matched and LC-MS was used to probe for salicylate presence between (D) plasmid-encoded EcN^{ATT} variants (n=6 replicates per group) and (E) selected variants between the EcN and EcN^{ATT} strains (n=3 replicates per group, 2-way ANOVA, Holm-Sidak post-test p=0.0001, **** p<0.0001). All samples were normalized to an internal isotope-labelled D4-salicylate standard. (F-G) 15-week-old Apc^{Min/+} mice were dosed with EcN^{ATT}-EntC-ColE1, (F) stool was collected at 48 h, homogenized, and plated on selective plates to quantify recoverable bacteria (n=3 mice per group, Er) and recoverable bacteria retaining the salicylate-encoded plasmid (Kan) and (G) urine was collected 24 h and 48 h after dosing and salicylate quantified using LC-MS in WT and Apc^{Min/+} mice (n=3 mice per group, ns=not significant, *, p=0.0228 2-Way ANOVA with Holm-Sidak post-test). All error bars represent S.E.M.

FIGS. 4A-4G show that treatment with EcN engineered to produce immunotherapeutics reduces adenoma burden and modifies the tumor-immune microenvironment. (A) Schematic of orally-delivered EcN probiotic engineered to lyse and produce immunotherapeutic proteins in situ (top) and schematic of dosing regimen (bottom). (B-C) 15-week-old Apc^{Min/+} mice were dosed twice within 3-4 days and then with PBS (Unt), EcN genomically encoding a lysis circuit (SLIC) or SLIC producing granulocyte-macrophage colony-stimulating factor (GM-CSF), and blocking nanobodies against PD-L1 and CTLA-4 targets (SLIC-3). 1 month after dosing, mice were sacrificed, intestines were bisected, Swiss-rolled, paraffin embedded, sectioned, stained with hemotoxin and eosin and quantified for (B) overall tumor area (black: female mice; gray: male mice) and (C) tumor area along the intestine, D, duodenum, PJ, proximal jejunum, DJ, distal jejunum, I, ileum (* p=0.0215, ** p=0.0008, *** P, 0.001, ns, not significant, ordinary one-way ANOVA test with Holm-Sidak multiple comparisons test, n=5-10 mice per group). (D) Representative H&E-stained histology images of SLIC and SLIC-3 treated mice. (E-G) Using immunohistochemical (IHC) techniques, intestinal tissue sections from each mouse groups were stained and quantified for (E) CD3⁺, (F) CD8⁺, and (G) Granzyme B-positive cells (n=3-4 mice per group, each dot represents a polyp, n=80-130 polyps per group, *** p=0.0002, **** p<0.0001 ordinary one-way ANOVA with Tukey post-test). Representative IHC images of all three markers are shown beside their respective plots with positive staining depicted as brown puncta in SLIC and SLIC-3-treated mice. Scale bars represent 200 μm. All error bars represent S.E.M.

### DETAILED DESCRIPTION OF THE INVENTION

While the present invention may be embodied in many different forms, disclosed herein are specific illustrative embodiments thereof that exemplify the principles of the invention. It should be emphasized that the present invention is not limited to the specific embodiments illustrated. Moreover, any section headings used herein are for organizational purposes only and are not to be construed as limiting the subject matter described.

Unless otherwise defined herein, scientific, and technical terms used in connection with the present invention shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like.

In addition, ranges provided in the specification and appended claims include both end points and all points between the end points. Therefore, a range of 1.0 to 2.0 includes 1.0, 2.0, and all points between 1.0 and 2.0.

The term “about” as used herein when referring to a measurable value such as an amount, a temporal duration, and the like, is meant to encompass variations of .+−.20%, .+−.10%, .+−.5%, .+−.1%, or .+−.0.1% from the specified value, as such variations are appropriate to perform the disclosed methods.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or lists of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either”, “one of”, “only one of”, “exactly one of”, when used in the claims, shall have its ordinary meaning as used in the field of patent law.

In the claims, as well as in the specification above, all transitional phrases such as “comprising”, “including”, “carrying”, “having”, “containing”, “involving”, “holding” and the like are to be understood to be open-ended, i.e., to mean including but not limited to. Only the transitional phrases “consisting of” and “consisting essentially of” shall be closed or semi-closed transitional phrases, respectively.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

The inventions described herein relate to orally-deliverable programmable bacteria cells (e.g., E. coli Nissle 1917 bacteria) that that produce diagnostic and therapeutic agent, resulting in sensitive detection of early CRC lesions and reduction of tumor burden through local induction of robust anti-tumor immunity as described hereinbelow.

### Programmable Bacteria Cells

In some embodiments of the inventions described herein, diagnostic agents and/or therapeutic agents are produced by one or more programmable bacterial cells. The programmable bacterial cells comprise heterologous nucleic acid sequences, which include one or more sequences that encode the diagnostic agents and/or therapeutic agents and sequences that encode a synchronized lysis circuit (i.e., a quorum-sensing gene, a nucleic acid encoding a lysis gene, a promoter, and a terminator contained on a single operon). By including a synchronized lysis circuit, the programmable bacterial cells are capable of lysing in response to one or more internal or external stimuli, such as achieving a certain concentration or cell density in a tumor microenvironment, thereby releasing the diagnostic agents and/or therapeutic agents and other cellular components into the surrounding environment (e.g., tumor microenvironment).

In some embodiments, the diagnostic agent is luciferase, salicylate, or a combination of both. In some embodiments, the therapeutic agent is an antibody that specifically binds to PD-L1, an antibody that specifically binds to CTLA-4, or a cytokine such as GM-CSF. In some embodiments, the programmable bacterial cells comprise one or more nucleic acids encoding a plurality of therapeutic agents.

The term “heterologous nucleic acid sequence” refers to a nucleic acid derived from a different organism that encodes for a protein and which has been recombinantly introduced into a cell, In some embodiments, the heterologous nucleic acid sequence is introduced by transformation in order to produce a recombinant bacterial cell. Methods for creating recombinant bacterial cells are well known to those of skill in the art. Such methods include, but are not limited to, different chemical, electrochemical and biological approaches, for example, heat shock transformation, electroporation, liposome-mediated transfection, DEAE-Dextran-mediated transfection, or calcium phosphate transfection. Multiple copies of the heterologous nucleic acid sequence (e.g., between 2 and 10,000 copies) may be introduced into the cell.

In some embodiments, the heterologous nucleic acid sequences are in a plasmid. In some embodiments, the heterologous nucleic acid sequences are in a single operon and are integrated into the genome of the programmable bacterial cells. In some embodiments, the programmable bacterial cells comprise at least one inducible promoter or non-constitutive promoter that is in operable linkage with one or more of the heterologous nucleic acid sequences.

As used herein, the term “promoter” means at least a first nucleic acid sequence that regulates or mediates transcription of a second nucleic acid sequence. A promoter may comprise nucleic acid sequences near the start site of transcription that are required for proper function of the promoter. As an example, a TATA element for a promoter of polymerase II type. Promoters of the present invention can include distal enhancer or repressor elements that may lie in positions from about 1 to about 500 base pairs, from about 1 to about 1,000 base pairs, from 1 to about 5,000 base pairs, or from about 1 to about 10,000 base pairs or more from the initiation site.

The term “inducible promoter” refers to an operable linkage between a promoter and a nucleic acid sequence, whereby the promoter mediates the nucleic acid transcription in the presence or absence of at least one specific stimulus. In some embodiments, the inducible promoter mediates transcription of a nucleic acid sequence in the presence or absence of at least one, two, three, four, or five or more stimuli. In some embodiments, the one or more stimuli are produced in whole or in part by the programmable bacterial cells. In some embodiments, the only stimulus of the promoter is the presence of a certain concentration or density of programmable bacterial cell found in the subject of a patient (e.g., in a tumor).

An “operable linkage” refers to an operative connection between nucleic acid sequences, such as for example between a control sequence (e.g., a promoter) and another nucleic acid sequence that codes for a protein i.e., a coding sequence. If a promoter can regulate transcription of an exogenous nucleic acid sequence, then it is in operable linkage with the gene.

In accordance with the purposes of the inventions described herein, the programmable bacterial cells are preferably non-pathogenic and colonize tumors. One of ordinary skill in the art would know how to attenuate pathogenic bacteria to create non-pathogenic bacteria. In some embodiments, the bacteria are attenuated by removing, knocking out, or mutating a virulence gene such as altering genetic components of the bacterial secretion system.

In some embodiments, the programmable bacterial cells belong to at least one genus selected from the group consisting of Salmonella, Escherichia, Firmicutes, Bacteroidetes, Lactobacillus, and Bifidobacteria. In some embodiments, the bacterial cells belong to more than one genus selected from the group consisting of Salmonella, Escherichia, Firmicutes, Bacteroidetes, Lactobacillus, and Bifidobacteria.

In some embodiments, the programmable bacterial cells belong to the genus Escherichia. In particular embodiments, the programmable bacterial cells are Escherichia coli Nissle 1917 (EcN) cells. In one embodiment, the EcN cells comprise a knockout of the clbA gene (EcNΔclbA).

Some aspects of this invention implicitly relate to culturing the programmable bacterial cells described herein. In some embodiments, a culture comprises the programmable bacterial cells and a medium, for example, a liquid medium, which may also comprise: a carbon source, for example, a carbohydrate source, or an organic acid or salt thereof; a buffer establishing conditions of salinity, osmolarity, and pH, that are amenable to survival and growth; additives such as amino acids, albumin, growth factors, enzyme inhibitors (for example protease inhibitors), fatty acids, lipids, hormones (e.g., dexamethasone and gibberellic acid), trace elements, inorganic compounds (e.g., reducing agents, such as manganese), redox-regulators (e.g., antioxidants), stabilizing agents (e.g., dimethyl sulfoxide), polyethylene glycol, polyvinylpyrrolidone (PVP), gelatin, antibiotics (e.g., Brefeldin A), salts (e.g., NaCl), chelating agents (e.g., EDTA, EGTA), and enzymes (e.g., cellulase, dispase, hyaluronidase, or DNase). In some embodiments, the culture may comprise an agent that induces or inhibits transcription of one or more genes in operable linkage with an inducible promoter, for example doxicycline, tetracycline, tamoxifen, IPTG, hormones, or metal ions. While the specific culture conditions depend upon the particular programmable bacterial cells, general methods and culture conditions for the generation of microbial cultures are well known to those of skill in the art.

### Therapeutic Methods and Compositions

The inventions described herein also encompass methods of treating a colorectal tumor in a subject comprising administering a therapeutically effective amount of programmable bacterial cells described herein to the subject, wherein the programmable bacterial cells comprise a nucleic acid encoding a therapeutic agent described herein, which capable of treating the colorectal tumor. The present disclosure also relates to methods of reducing the rate of proliferation of a colorectal tumor cell comprising delivering a programmable bacterial cell described herein to the colorectal tumor cell. The present disclosure also relates to methods of killing a colorectal tumor cell comprising delivering a programmable bacterial cell described herein to the colorectal tumor cell.

As used interchangeably herein, “treatment” or “treating” or “treat” refers to all processes wherein there may be a slowing, interrupting, arresting, controlling, stopping, alleviating, or ameliorating symptoms or complications, or reversing of the progression of colorectal cancer, but does not necessarily indicate a total elimination of all disease or all symptoms. Non-limiting examples of treatment include reducing the rate of growth of a colorectal tumor or colorectal cancer cell disease, reducing the size of a tumor, or preventing the metastases of a tumor.

Programmable bacterial cells described herein are preferably administered in one or more therapeutically effective doses. As used herein the terms “therapeutically effective dose” means the number of cells per dose administered to a subject in need thereof that is sufficient to treat the hyperproliferative disorder. In some embodiments, a therapeutically effective dose can be at least about 1×10⁴ cells, at least about 1×10⁵ cells, at least about 1×10⁶ cells, at least about 1×10⁷ cells, at least about 1×10⁸ cells, at least about 1×10⁹ cells, or at least about 1×10¹⁰ cells.

In some embodiments, programmable bacterial cells may be delivered to a subject in the form of a pharmaceutical composition, which may comprise one or more pharmaceutically acceptable carriers, diluents, or excipients. Pharmaceutical compositions may be formulated as desired using art recognized techniques. Various pharmaceutically acceptable carriers, which include vehicles, adjuvants, and diluents, are readily available from numerous commercial sources. Moreover, an assortment of pharmaceutically acceptable auxiliary substances, such as pH adjusting and buffering agents, tonicity adjusting agents, stabilizers, wetting agents, and the like, are also available. Certain non-limiting exemplary carriers include saline, buffered saline, dextrose, water, glycerol, ethanol, and combinations thereof. Pharmaceutical compositions may be frozen and thawed prior to administration or may be reconstituted in WFI with or without additional additives (e.g., albumin, dimethyl sulfoxide). Programmable bacterial cells described herein are preferably formulated for oral administration, but other routes of administration known in the art may be utilized.

Particular dosage regimens, i.e., dose, timing, and repetition, will depend on the particular subject being treated and that subject's medical history. Empirical considerations such as pharmacokinetics will contribute to the determination of the dosage. Frequency of administration may be determined and adjusted over the course of therapy and is based on reducing the number of tumor cells or tumor mass, maintaining the reduction of such tumor cells or tumor mass, reducing the proliferation of tumor cells or an increase in tumor mass, or delaying the development of metastasis. A therapeutically effective dose may depend on the mass of the subject being treated, his or her physical condition, the extensiveness of the condition to be treated, and the age of the subject being treated.

### Articles of Manufacture

The inventions disclosed herein also encompass articles of manufacture useful for treating a colorectal tumor comprising a container comprising programmable bacterial cells described herein, or a pharmaceutical composition comprising the same, as well as instructional materials for using the same to treat the colorectal tumor. In some embodiments, the articles of manufacture are part of a kit that comprises a bacterial culture vessel and/or bacterial cell growth media.

### EXAMPLES

The following examples have been included to illustrate aspects of the inventions disclosed herein. In light of the present disclosure and the general level of skill in the art, those of skill appreciate that the following examples are intended to be exemplary only and that numerous changes, modifications, and alterations may be employed without departing from the scope of the disclosure.

### Example 1

### Strains and Plasmids

All bacterial strains used were luminescent (integrated luxCDABE cassette) so they could be visualized with the In Vivo Imaging System (IVIS). The EcNΔclbA strain was engineered using the lambda-red recombineering method. The salicylate-encoding plasmid was constructed using Gibson assembly methods or restriction enzyme-mediated cloning methods whereby pchA and pchB genes were cloned onto a high-copy origin plasmid and driven by the lac promoter. The SLIC and SLIC-3 strains were constructed as previously described.

### Example 2

### Bacterial Preparation for Oral Administration

Overnight cultures of EcN-lux were diluted 1:100 into LB with 50 ng/ml erythromycin and cultured to an OD₆₀₀ of ˜0.5 on a shaker at 37° C. Bacteria were collected by centrifugation at 3000 r.c.f., washed three times with sterile PBS, resuspended in sterile ice-cold PBS with a total of 100-200 μL dosed at orally at a concentration of ˜1010 CFU/mL. SLIC strains were prepared as previously described. Briefly, growth media for SLIC and SLIC-3 strains also contained 0.2% glucose to suppress premature lysis in culture. Additionally, SLIC-3 strains were grown with 50 mg/mL kanamycin.

### Example 3

### Organoid Culture

Mouse CRC Braf^{V600E}; Tgfbr^{2Δ/Δ}; Rnf43^{Δ/Δ}/Znrf^{3Δ/Δ}; p16 Ink4a^{Δ/Δ} (Braf^{V600E}ΔTRZI, MSS CRC) organoids were generated using CRISPR/Cas9 genome engineering and expanded for injection into mice in matrigel culture as previously described. Culture medium was Advanced Dulbecco's modified Eagle medium/F12 (Life Technologies) supplemented with 1× gentamicin/antimycotic/antibiotic (Life Technologies), 10 mM HEPES, 2 mM GLUTAMAX®, 1×B27 (Life Technologies), 1×N2 (Life Technologies), 50 ng/ml mouse recombinant EGF (Peprotech), 100 ng/ml mouse recombinant noggin (Peprotech), 10 ng/mL human recombinant TGF-β1 (Peprotech). Immediately after each split, organoids were cultured in 10 μM Y-27632 (In Vitro Technologies), 3 μM iPSC (Calbiochem Cat #420220), 3 μM GSK-3 inhibitor (XVI, Calbiochem, #361559) for the first 3 days.

### Example 4

### Orthotopic Mouse Model of CRC

All animal experimentation related to the orthotopic CRC model was approved by the institutional animal ethics committee (SAM-319, SAM-20-031, SAM-21-041). Orthotopic injections to generate distal colon tumors were undertaken as previously described. In brief, NOD. (g-Prkdc^{scid}Il2rg^{tm1il}/SzJ (NSG) mice (male and female, 6-12 weeks old) were obtained from the SAHMRI Bioresources facility and housed under SPF conditions. Digested MSS or MSI organoid clusters (equivalent to ˜150 organoids) were resuspended in 20 μL 10% GFR matrigel 1:1000 India ink, 10 μM Y-27632 in PBS and injected into the mucosa of the distal colon of anesthetized NSG mice using colonoscopy-guided orthotopic injection (2 injection sites/mouse). Injection sites were monitored by weekly colonoscopy. EcN administration began once the tumors were clearly established (grade 3 to 4 on the Becker Scale), at 4-6 weeks post organoid injection. MSS tumors were treated with broad-spectrum antibiotic treatment to generate gut dysbiosis in specific groups involved administration of 0.5 g/L neomycin and 1 g/L ampicillin in ad libitum drinking water for 5 days. This was halted 6 hours prior to EcN administration.

### Example 5

### Apc^{Min/+} Mouse Model of CRC

All animal experimentation related to the Apc^{Min/+} mouse model of CRC was approved by the Institutional Animal Care and Use Committee (Columbia University, protocols AC-AAAN8002 and AC-AAAZ4470). All mice were regularly monitored and euthanized based on veterinarian recommendation or when they reached ˜20 weeks of age. In all therapeutic studies wild-type (WT) littermates of Apc^{Min/+} on the C57BL/6 background were used and both males and females were treated and evenly distribute among groups. For diagnostic studies, Apc^{Min/+} mice were purchased from Jackson Laboratories and C57BL/6 mice were used as WT.

### Example 6

E. coli Nissle (EcN) 1917 Biodistribution

To quantify the EcN-lux derived luciferase signal in mouse models of CRC, a XENOGEN® in vivo imaging system (IVIS®) Spectrum Imager (Perkin Elmer Inc). Following necropsy, individual mouse tissues were collected into individual wells of a 6-well plate, weighed and background (stage alone) subtracted average radiance (photons/s/cm²/sr) measurements were used to correct for the area being measured which differed for each tissue analyzed.

Excised mouse tissues were placed aseptically into 5 mL 20% glycerol in PBS and homogenized in GENTLEMACS™ cell dissociator C tubes, one tissue per tube using program C. 100 μL of each tissue homogenate glycerol stock was serially diluted 1:100 six times. 10 μL of each dilution was spotted onto an LB agar plate with 50 μg/mL erythromycin selection with 5 technical replicates. Plates were incubated at 37° C. overnight (16 hours). Colony forming units (CFU) were calculated for each sample normalized to the weight of tissue input to generate CFU/g tissue. To generate CFU/g stool, one pellet of stool was placed into a 1.5 mL microcentrifuge tube and manually homogenized in PBS with a pipette tip and rigorous pipetting. Serial dilutions were spotted onto an LB agar place with 50 μg/mL erythromycin and incubated at 37° C. overnight. CFU was normalized to weight.

### Example 7

### Clinical Trial Design

This study was an interventional, double-blind, dual-center, prospective clinical trial (WHO Universal Trial Number U1111-1225-7729, ANZCTR number ACTRN12619000210178). The study was approved by the Human Research Ethics Committee of the Central Adelaide Local Health Network (HREC/18/CALHN/751) to meet the requirements of the National Statement on Ethical Conduct in Human Research in accordance with the Declaration of Helsinki for medical research involving human subjects. The study objective was to evaluate the colonization of matched normal and neoplastic bowel tissue by the probiotic E. coli Nissle.

Adult participants undergoing routine colonoscopy or surgical resection for primary colorectal cancer were recruited from St. Andrew's Hospital and Royal Adelaide Hospital, Adelaide (N=36). Written, informed consent was provided before participants were assigned to take either 2 tablets (10⁹ CFU) per day of non-genetically modified EcN (MUTAFLOR®) or placebo for 14 days, prior to their procedure. Patients and treating physicians were blind to active or placebo status. Participants were provided with probiotic tablets and instructed to begin 14 days prior to resection. On the morning of resection investigators verbally confirmed with the participants that they had taken the entire probiotic course, stopping on the day before surgery. Tere were no alternate methods employed to validate treatment uptake, other than PCR detection of EcN DNA sequence in microbial cultures from tissue samples. Mucosal biopsies (colonoscopy) or surgical resection samples from normal and neoplastic tissue were collected from each participant at the time of their procedure. Participants were excluded if they took probiotics or antibiotics during the trial period.

Eight participants withdrew from the study primarily due to treatment plan change, i.e., no longer undergoing surgery and 2 participants did not have sufficient tumor tissue present to sample from surgery and were excluded. The trial was terminated early after accrual of 35 of the planned 110 participants due to COVID-19 related restrictions and concerns colibactin-expressing E. coli may be pro-carcinogenic. The planned primary outcome of neoplastic colonization status of the probiotic in CRC patient tissues is reported herein, the secondary outcome of microbiome analyses associated with colonization status was discontinued due to smaller than expected sample size.

### Example 8

### Human Tissue Sample Analysis

Tissue samples were weighed and collected in sterile 20% glycerol in PBS. Tissue was homogenized in GENTLEMACS™ C Tubes (Miltenyi Biotec, 130-093-237), with a GENTLEMACS™ Dissociator (Miltenyi Biotec, 130-093-235), program E. Aliquoted, homogenized tissue was stored at −80° C. until further use. For culture enrichment, the equivalent of 10 mg of human tissue in homogenate was added to 1.2 mL of LB broth/sample and incubated with shaking at 37° C. for 24 hours. Culture OD was monitored hourly for the first 14 hours to ensure exponential growth, samples from three patients were excluded due to inability to attain log phase cultures from tissue homogenates. 1 mL of saturated culture at 24 hours was centrifuged at 10,000×g to collect cells and DNA extracted from cell pellet using DNEASY® POWERSOIL® Pro kit (Qiagen, 47016).

### Example 9

Development of E. coli Nissle 1917 Strain Specific PCR Assay for Human Samples

E. coli Nissle pMUT2 primers ECN7/8 and 9/10 were tested to detect E. coli Nissle in mouse fecal samples but they generated unacceptable false positives using gDNA isolated from human tissue samples from untreated patients. Alignment of PCR primer sets ECN7/8 or 9/10 against DNA sequences using Primer-BLAST suggested that Edwardsiella and Plesiomonas contain highly related sequences potentially also found in the human gut that may cause false positive calls via PCR assay using these primers. To avoid this confounding amplification during E coli Nissle 1917 detection, a different nested PCR strategy was devised to boost specificity and sensitivity for use with human samples using DNA sequence from pMUT2 unique to E. coli Nissle 1917 in comparison with human gut microbiota sequences. The external 283 bp amplicon spans the unique pMUT2 DNA region: ext-F 5′ TCGCGAACGTTAAATAATCATC (SEQ ID NO: 1); ext-R 5′ TCTGTTTTAGATAAGGCCATGTCTTC (SEQ ID NO: 2), and was amplified from 50 ng DNA input using KAPA Probe qPCR Master Mix (Roche, KK4716) with PCR conditions: denaturation 95° C. for 20 seconds; 10 cycles of 95° C. for 1 second, 60° C. for 20 seconds, and 72° C. for 25 seconds. Then 1 μL of this reaction was used as the template for the second 114 bp nested primer/probe-based assay. Nested primer and probe sequences were: int-F 5′ ACCCATCGATACCAAATGTATGT (SEQ ID NO: 3); int-R 5′ TCAATGCGTACTCGACTATTCAAA (SEQ ID NO: 4); probe 5′/56-FAM/CCCGCAGAT/ZEN/CACTGACCTCAATACA (SEQ ID NO: 5)/31ABKFQ/using KAPA Probe qPCR Master Mix with PCR conditions as follows: 95° C. for 20 seconds, 40 cycles of 9° C. for 1 second, 60° C. for 20 seconds, and 72° C. for 25 seconds. For 16S PCR, standard KAPA SYBR (non-nested) qPCR Master Mix (Roche, KK4602) with primers reported to amplify a 466 bp amplicon covering 331-797 of the E. coli 16S TRNA gene 16S-F 5′ TCCTACGGGAGGCAGCAGT (SEQ ID NO: 6) and 16S-R 5′ GGACTACCAGGGTATCTAATCCTGTT (SEQ ID NO: 7). E. coli Nissle 1917 PCR standards were generated from serially diluted DNA isolated from exponentially growing cultures from crushed MUTAFLOR® capsule in LB at 37° C., with CFU determined by plating of matched samples on LB agar plates.

### Example 10

### Salicylate Metabolite Detection

For in vitro samples, overnight cultures of EcN-SA were OD₆₀₀ matched to be 1. Cultures were then centrifugated at 3000×g and 1 mL of the supernatant was collected and stored at −80° C., the rest was decanted, and the pellet was stored at −80° C. as well until analysis. To 1 mL of supernatants, 1000 μL of extraction solvent (MeOH/MeCN/H₂O containing 0.1 mg/mL of D4-salicylate) was added. Samples were homogenized using a Bead Ruptor 4 at speed 4 for 10 seconds for 5 cycles. The homogenized samples were centrifuged for 5 min at 14,000×g. Then, the supernatant was removed and dried on the Gene Vac for 5 hours and re-suspended in 200 μL of MeCN/H₂O before LC-MS analysis. A quality control (QC) sample was prepared by combining 20 μL of each sample to assess the reproducibility of the features through the runs.

Chromatographic separation was carried out at 40° C. on Acquity UPLC® BEH C18 column (2.1× 50 mm, 1.8 μm) over a 7-min gradient elution. Mobile phase A consisted of water and mobile phase B was acetonitrile both containing 0.1% formic acid. After injection, the gradient was held at 99% mobile phase A for 0.5 min. For the next 4 min, the gradient was ramped in a linear fashion to 50% B and held at this composition for 1 min. The eluent composition returned to the initial condition in 0.1 min, and the column was re-equilibrated for an additional 1 min before the next injection was conducted. The flow rate was set to 450 μL/min and Injection volumes were 2 μL using the flow-through needle mode in the negative ionization mode. The QC sample was injected between the samples and at the end of the run to monitor the performance and the stability of the MS platform.

The SYNAPT® G2-Si mass spectrometer (Waters, Manchester, U.K.) was operated in the negative electrospray ionization (ESI) modes. A capillary voltage of −1.5 kV and a cone voltage of 30 V was used. The source temperature was 120° C., and desolvation gas flow was set to 850 L/hr. Leucine enkephalin was introduced to the lock mass at a concentration of 2 ng/μL (50% ACN containing 0.1% formic acid), and a flow rate of 10 μL/min for mass accuracy and reproducibility. The data was collected in duplicates in the centroid data-independent (MSE) mode over the mass range m/z 50 to 650 Da with an acquisition time of 0.1 seconds per scan. The QC sample, D4-salicylate, and salicylate standards were also acquired in enhanced data-independent ion mobility (IMS-MSE) in negative modes for the structural assignment. The ESI source settings were the same as described above. The traveling wave velocity was set to 650 m/s and the wave height was 40 V. The helium gas flow in the helium cell region of the ion-mobility spectrometry (IMS) cell was set to 180 mL/min to reduce the internal energy of the ions and minimize fragmentation. Nitrogen as the drift gas was held at a flow rate of 90 mL/min in the IMS cell. The low collision energy was set to 4 eV, and the high collision energy was ramped from 25 to 50 eV in the transfer region of the T-Wave device to induce fragmentation of mobility-separated precursor ions.

All raw data files were converted to netCDF format using DataBridge tool implemented in MASSLYNX® software (Waters, version 4.1). Then, they were subjected to peak-picking, retention time alignment, and grouping using XCMS package (version 3.2.0) in R (version 3.5.1) environment. Technical variations such as noise were assessed and removed from extracted features' list based on the ratios of average relative signal intensities of the blanks to QC samples (blank/QC>1.5). Also, peaks with variations larger than 30% in QCs were eliminated. The detected signal intensity of salicylate in the samples were normalized to the signal intensity of labeled D4-salicylate. Group differences in measured salicylate levels were calculated using the Welch t-test, p-value<0.05 in GRAPHPAD PRISM®.

Urine samples were collected from mice 24 hours after EcN-salicylate strain variant (EcN-SA) oral dosing and frozen at −80° C. for later LC-MS analysis. For LC-MS analysis, urine samples were thawed on ice and polar metabolites were extracted with addition of 100% ice-cold LC-MS grade methanol with 0.1 M formic acid (4:1 ratio of extraction solvent volume to urine). Samples were then vortexed and centrifuged at 20,000×g for 10 minutes at 4° C. Supernatants were then transferred to clean LC-MS tubes and loaded onto the LC-MS autosampler, which was temperature-controlled at 4° C.

Targeted analysis was performed as previously described. In brief, samples were separated by liquid chromatography on an AGILENT 1290 INFINITY® LC system by injection of 3 μL of extract through an Agilent INFINITYLAB® POROSHELL® 120 HILIC-Z, 2.1×150 mm, 2.7 μm (Agilent Technologies) column heated to 50° C. Solvent A (100% water containing 10 mM ammonium acetate, 5 mM INFINITYLAB® Deactivator Additive and adjusted to pH 9 using ammonium hydroxide) and Solvent B (85% acetonitrile/15% water containing 10 mM ammonium acetate, 5 mM INFINITYLAB® Deactivator Additive and adjusted to pH 9 using ammonium hydroxide) were infused at a flow rate of 0.250 mL/min. The 26-min normal phase gradient was as follows. 0-2 min, 96% B; 5.5-8.5 min, 88% B; 9-14 min, 86% B, 17 min, 82% B; 23-24 min, 65% B; 24.5-26 min, 96% B; followed by a 10-min post-run at 96% B. Acquisition was performed on an Agilent 6230 TOF mass spectrometer (Agilent Technologies) using an Agilent Jet Stream electrospray ionization source (Agilent Technologies) operated at 3,500 V Cap and 0 V nozzle voltage in extended dynamic range, negative mode. The following settings were used for acquisition: The sample nebulizer set to 35 psi with sheath gas flow of 12 L/min at 350° C. Drying gas was kept at 350° C. at 13 L/min. Fragmentor was set to 90 V, with the skimmer set to 45 V and Octopole Vpp at 750 V. Samples were acquired in centroid mode at 1 spectra/s for m/z values from 50-1,700.

Raw data was acquired from the instrument and analyzed using previously described open-source XCMS software. Metabolites were identified from (m/z, rt) pairs by retention time comparison with authentic standards.

### Example 11

### Histology

For the Apc^{Min/+} mouse model, all intestinal tissue was excised with the caecum removed and tissue was bisected such that there were 5 total sections: duodenum, proximal jejunum, distal jejunum, ileum, and colon. Intestines were flushed with PBS, splayed open, Swiss-rolled, and fixed overnight in 4% paraformaldehyde. After 24 hours the Swiss rolls were switched to 70% ethanol and sent for histology services at Histowiz, where they were paraffin-embedded, sectioned, and stained with either H&E or specific immunohistochemistry markers (HA-Tag C29F4 #3724 from Cell Signaling Technology; Granzyme B, CD3 Abcam 16669; CD8 catalog #CST98941 clone D4W2Z). Tumor sizes and IHC quantification were determined using FIJI software image analysis tools.

For the orthotopic CRC transplant model, mice were intraperitoneally injected with pimonidazole-HCl (HYPOXYPROBE®, 60 mg/kg) or PBS negative control for HYPOXYPROBE® staining, 1 hour prior to euthanasia. Tumor, nearby adjacent colonic tissue, and kidneys were collected at necropsy, rinsed in PBS, and fixed overnight in formalin prior to dehydration in 70% ethanol and paraffin-embedding. An additional positive control tumor sample for optimizing ISH staining was generated by intratumoral injection of EcN-lux into a dissected tumor sample from a PBS treated mouse ex vivo, prior to fixation. Formalin-fixed and paraffin-embedded tissue sections were stained with H&E. Serial sections were also subjected to co-immunofluorescence staining against HYPOXYPROBE® (cat #HP12-200 Kit, 1:200 dilution) and lipopolysaccharide (LPS, cat #HM6011-100UG, 1:500 dilution) or chromogenic in situ hybridization using RNASCOPE® technology (RNASCOPE® 2.5 Detection Kit, Advanced Cell Diagnostics) following the manufacturer's instructions with a custom probe to detect the lux transcript in EcN-lux or negative control probe, DapB (target region 414-862; catalogue number 310043). Briefly for ISH, tissue sections were baked in a dry oven (HYBEZ™ II Hybridization System, ACD) at 60° C. for 1 hour and deparaffinized, followed by incubation with hydrogen peroxide (Lot #322000, ACD) and targeted retrieval (Lot #322330, ACD). Slides were incubated with relevant probes for 2 hours at 40° C., followed by successive incubations with Amp1 to 6 reagents. Staining was visualized with DAB. For IF studies, sections were treated with blocking buffer (X0909, Dako) for 30 min, incubated with the indicated primary antibodies overnight at 4° C., and washed with PBS. Sections were then incubated with ALEXA FLUOR® 488/594-conjugated secondary antibodies (1.200 dilution, Thermo Fisher Scientific) for 1 h at room temperature. The sections were then mounted with VECTASHIELD® antifade mounting medium (Cat #H-1000-10, Vector Laboratories), and fluorescence was examined using a confocal laser-scanning microscope (FV3000, Olympus).

Resected human CRC samples were fixed overnight in formalin prior to dehydration in 70% ethanol and paraffin-embedding. Formalin-fixed and paraffin-embedded tissue sections were stained with H&E. Serial sections were also subjected to immunofluorescence staining against lipopolysaccharide (LPS, cat #HM6011-100UG, 1:500 dilution). Sections were treated with blocking buffer (X0909, Dako) for 30 min, incubated with the primary antibody overnight at 4° C., and washed with PBS. Sections were then incubated with ALEXA FLUOR® 488-conjugated secondary antibody (1:200 dilution, Thermo Fisher Scientific) for 1 hour at room temperature. The sections were mounted with VECTASHIELD® antifade mounting medium (Cat #H-1000).

### Example 12

Adenoma Colonization by E. coli Nissle 1917 (EcN) in a Genetically-Engineered Mouse Model of CRC Predisposition

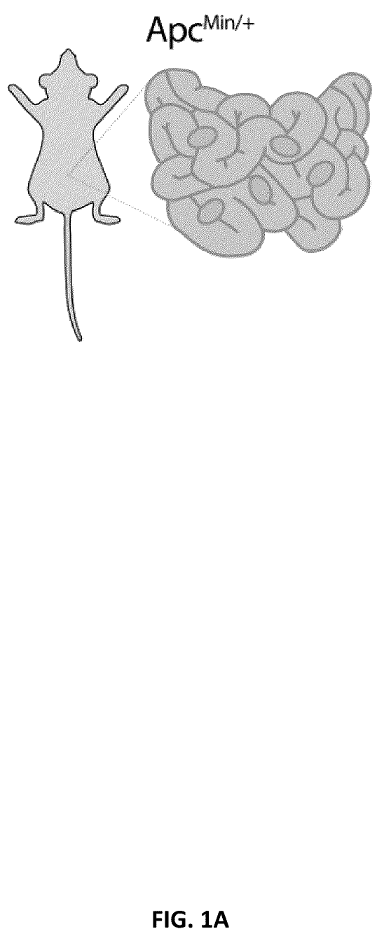

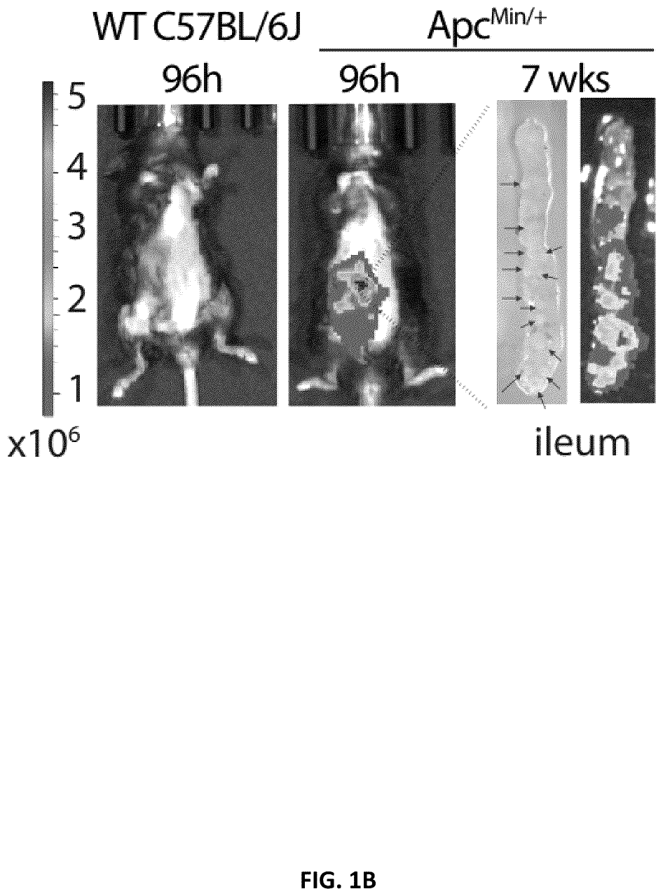

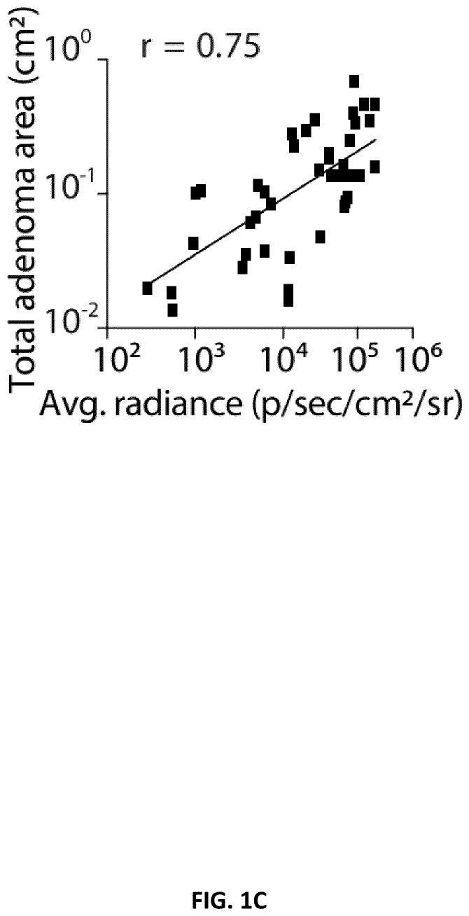

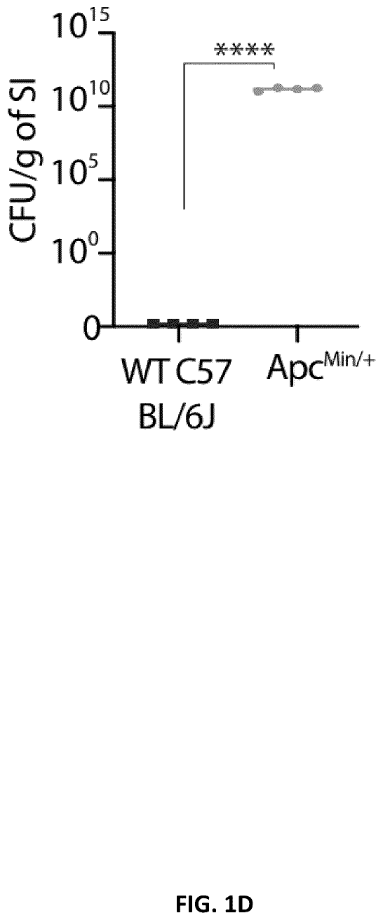

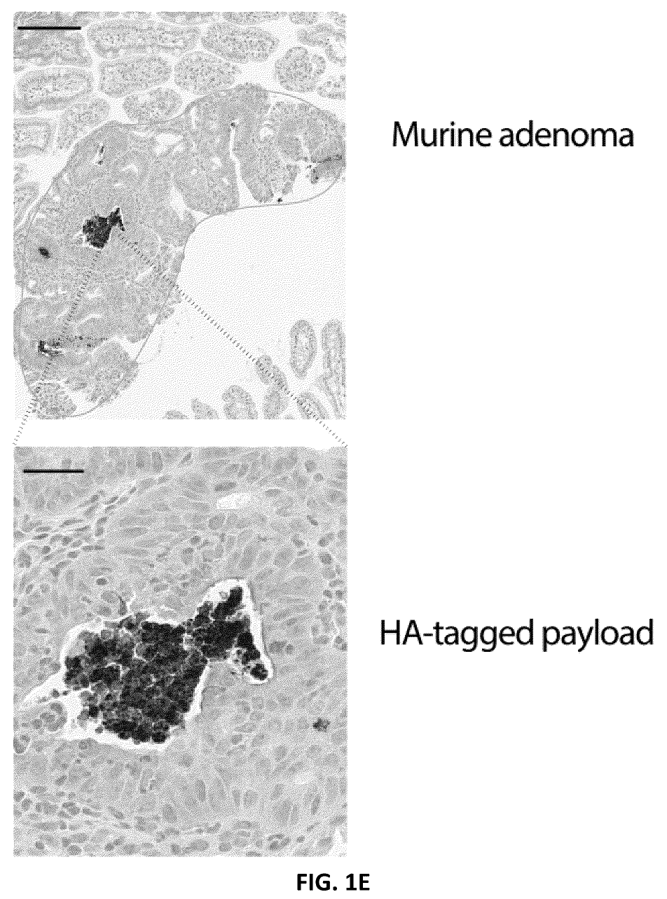

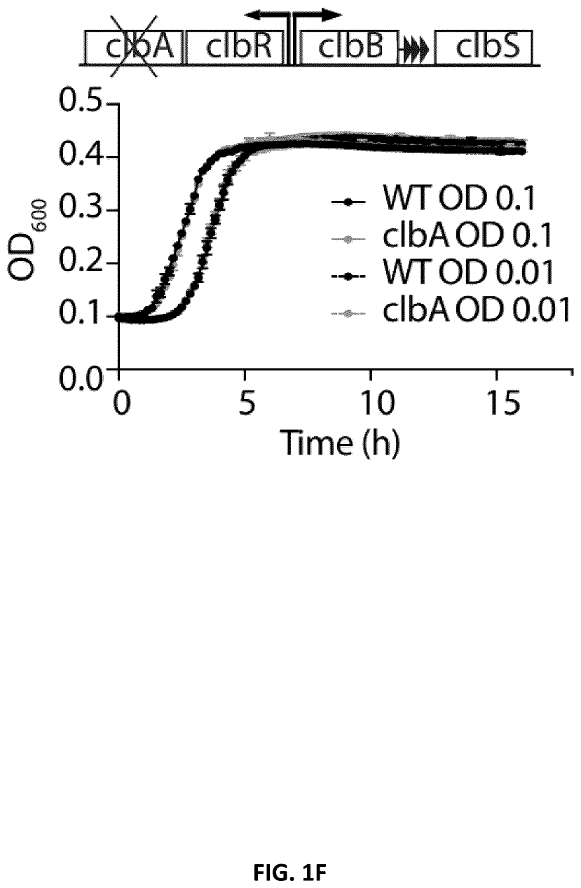

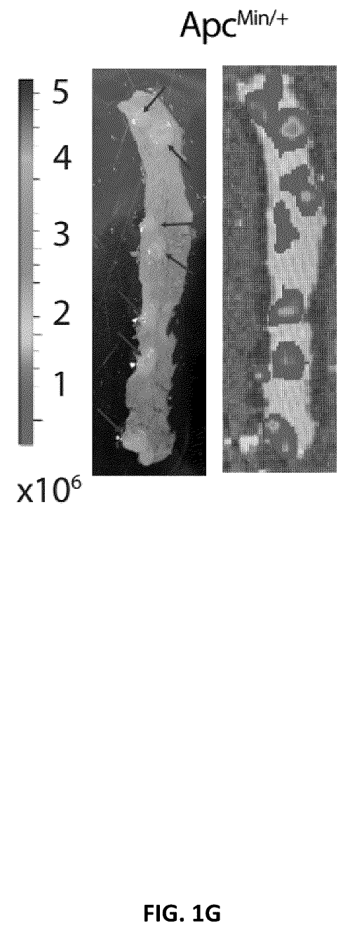

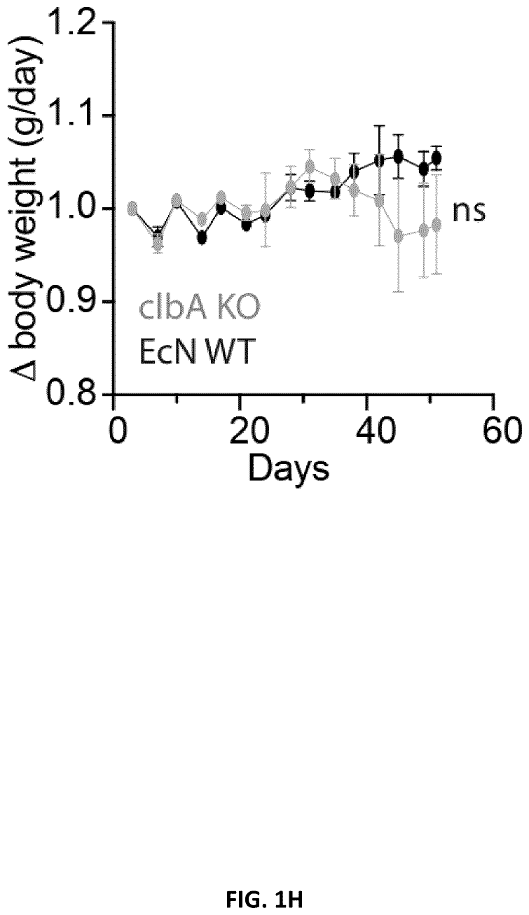

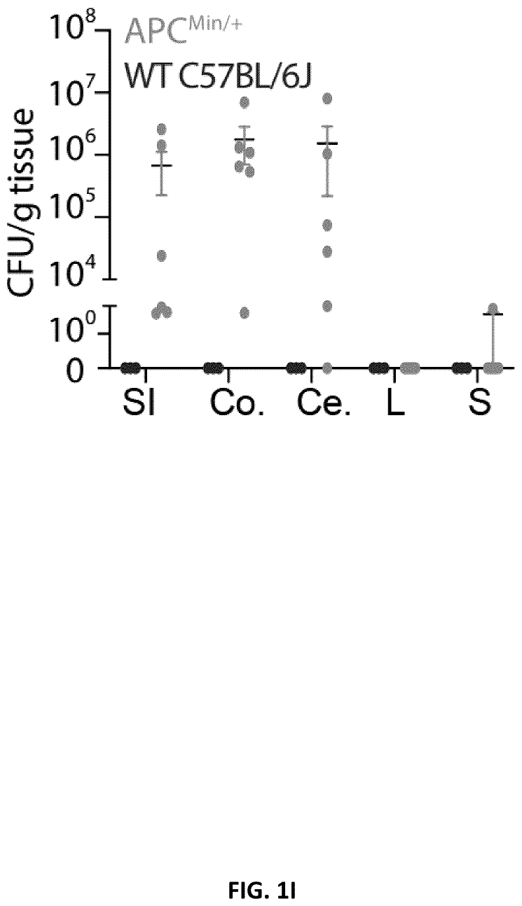

CRC precursor lesions were modeled using Apc^{Min/+} in mice, a well-established mouse model of familial adenomatous polyposis (FAP), whereby mice spontaneously develop adenomas throughout their intestinal tract. This model is driven by an APC gene mutation—an initiating genetic mutation observed in ˜80% of human CRC (FIG. 1A). Adenoma colonization was explored by orally-delivering EcN encoding a genomically-integrated luxCDABE cassette (EcN-lux) to Apc^{Min/+} mice with an intact immune system and microbiome. In vivo imaging of mice dosed with EcN-lux showed elimination of bioluminescent bacteria in healthy wild-type (WT) mouse gut, but retention in the Apc^{Min/+} mouse gut for up to 7 weeks after oral administration (FIG. 1B). Enrichment of EcN-lux in neoplasia was further demonstrated with ex vivo imaging of intestinal tissue, where bioluminescence co-localized with visible macroadenomas and generally more bioluminescence was observed in the distal small intestine where adenoma burden was the greatest (FIG. 1C). Subsequent plating of homogenized intestinal tissue on antibiotic-selective Luria broth (LB) plates specific for EcN-lux indicated that no detectable EcN-lux could be recovered from wild-type mouse tissue, suggesting that a long-term niche is not formed in the gut unless neoplastic tissue is present (FIG. 1D). To further investigate bacteria localization, EcN-lux was engineered to release a human influenza hemagglutinin (HA)-tagged protein under control of a lysis circuit and orally delivered to Apc^{Min/+} mice. Four weeks after oral delivery, mice were sacrificed and their intestinal tissue was probed for positive HA signal. Dark positive stains of EcN-tagged payloads were identified in adenomas of varying sizes, suggesting functional delivery of payloads released by the lysis circuit following oral delivery of the engineered strain (FIG. 1E).

Due to increased concerns of colibactin-producing bacteria like EcN being pro-carcinogenic, colibactin production was disrupted by deleting the clbA gene (EcNΔclbA) and observed no changes in bacterial growth kinetics at multiple seeding densities compared to the parent EcN strain (FIG. 1F). Neoplasia colonization was explored by orally-delivering EcNΔclbA-lux to Apc^{Min/+} mice. Observing co-localization of bioluminescent bacteria with macroadenomas (FIG. 1G) suggested that colonization does not rely on the presence of the clbA gene or an intact colibactin-encoding operon. In Apc^{Min/+} mice treated with either the EcN-lux or EcNΔclbA-lux strain, no notable differences in mouse body condition and weight were observe (FIG. 1H). Forty-eight hours post-dosing, the liver, spleen, cecum, colon, and the small intestine from each mouse were harvested and plated on antibiotic-selective LB plates to recover EcNΔclbA-lux. While EcNΔclbA-lux was found abundantly in the intestines of polyp bearing animals compared to healthy controls, fewer to no bacteria were recovered from the liver and spleen of Apc^{Min/+} mice or from wild-type control mice, demonstrating minimal off-target localization (FIG. 1I).

### Example 13

### Tumor Colonization by EcN in Orthotopic Mouse Models of CRC

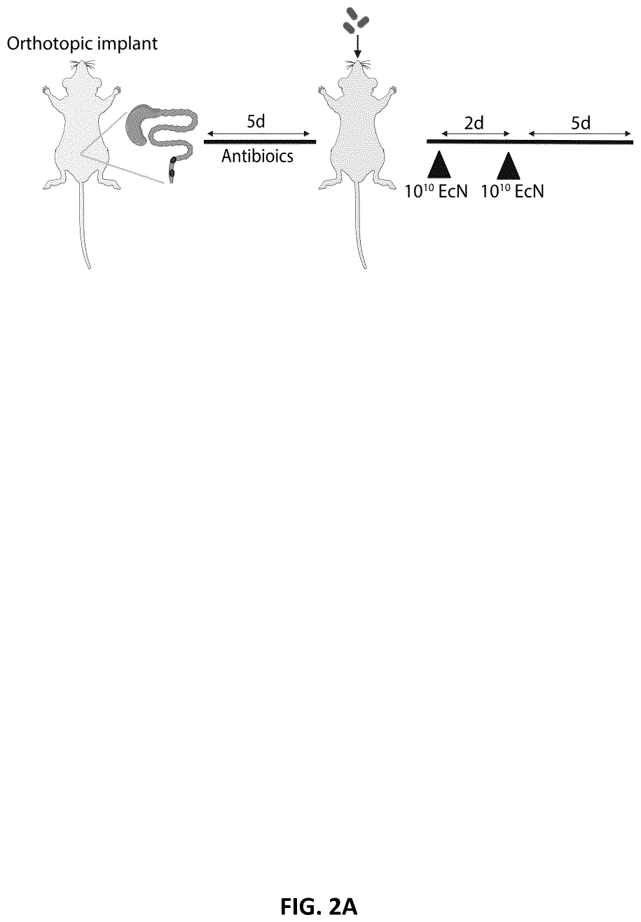

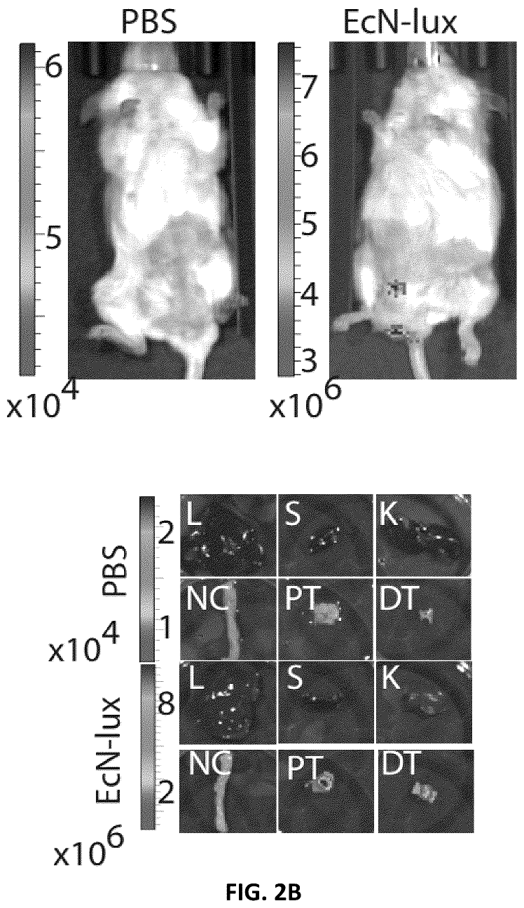

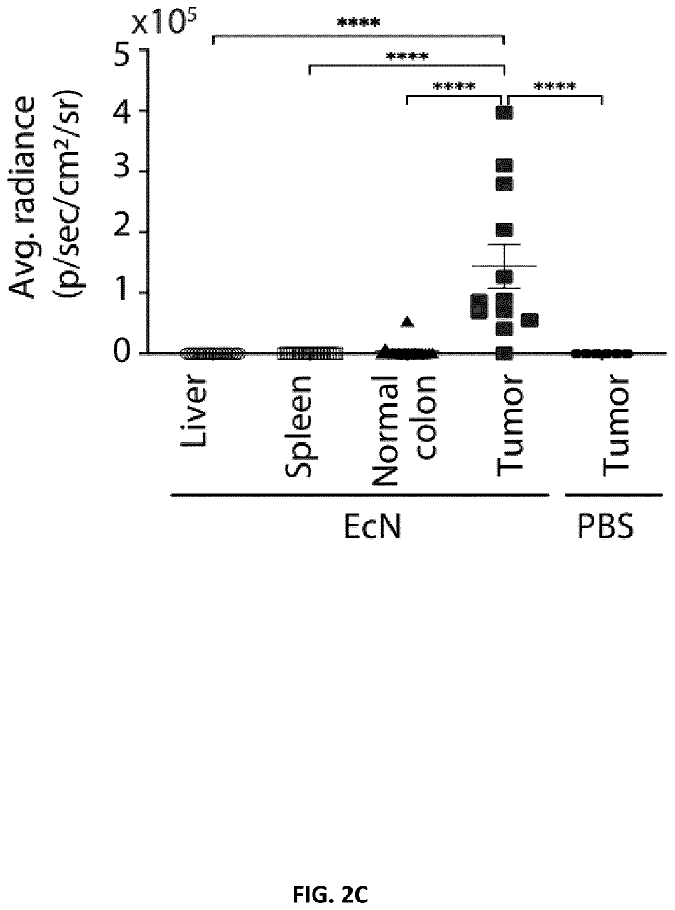

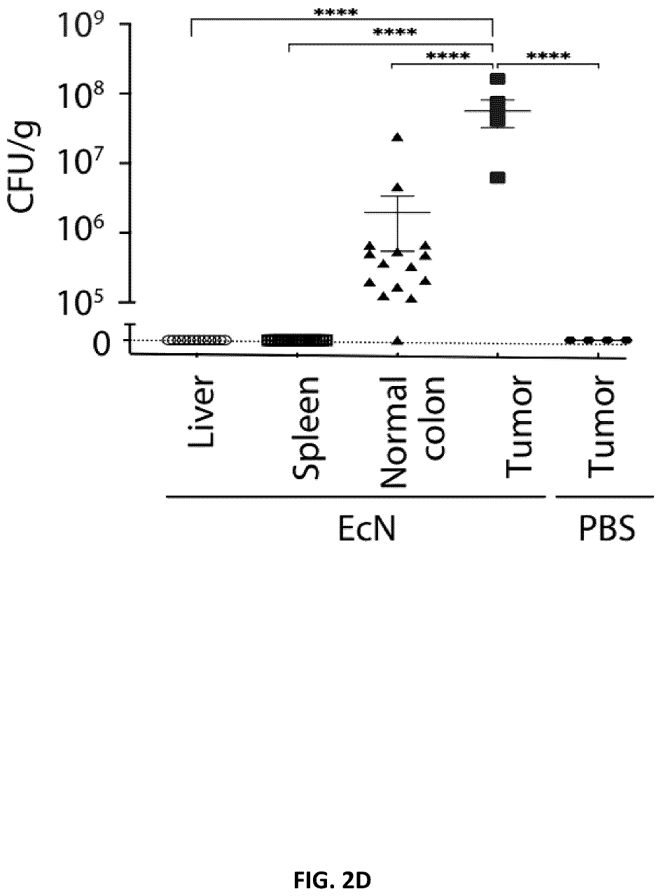

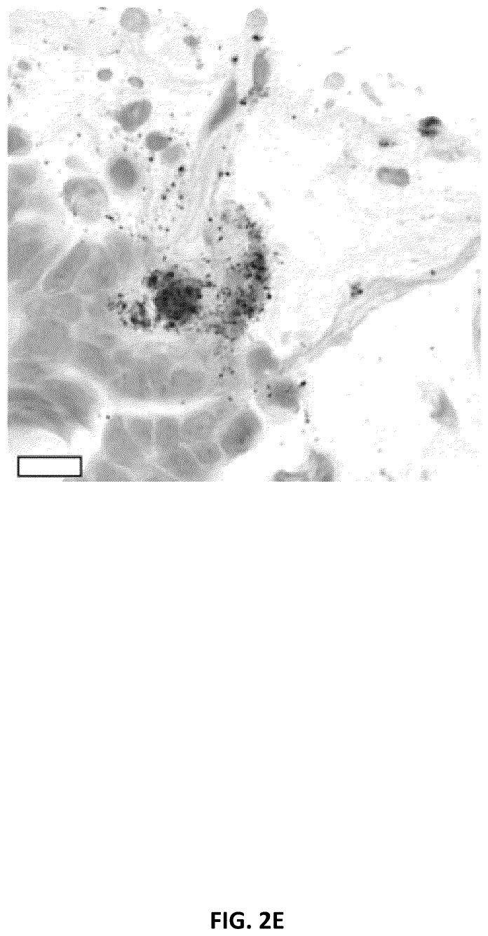

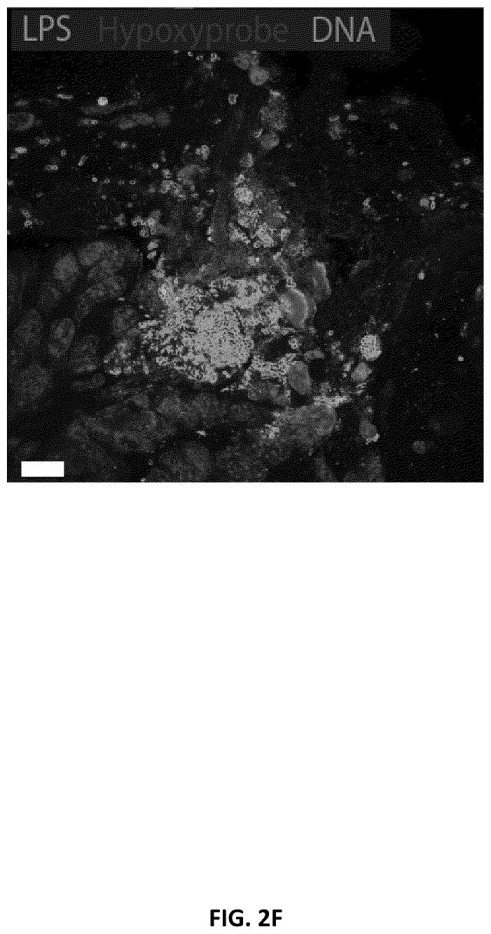

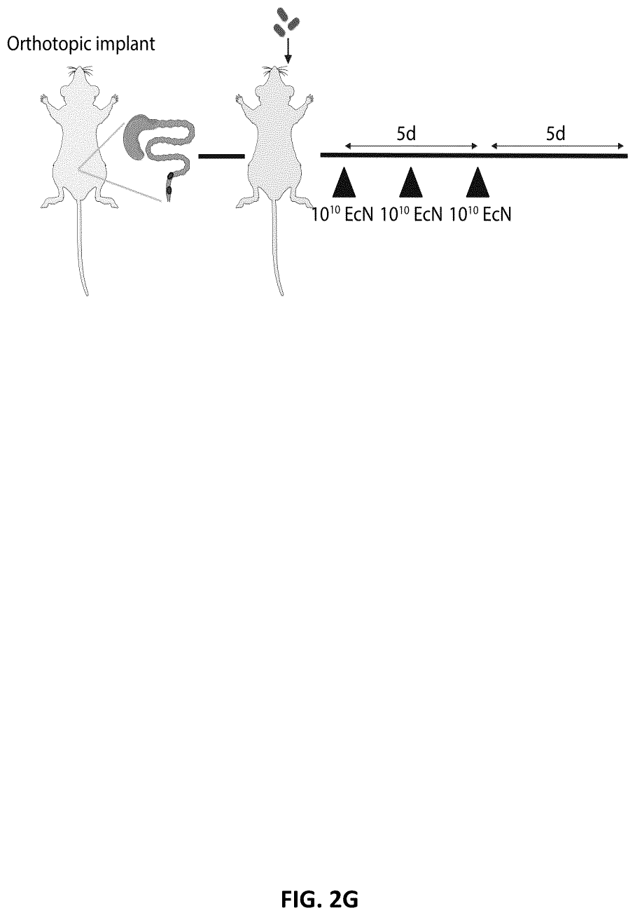

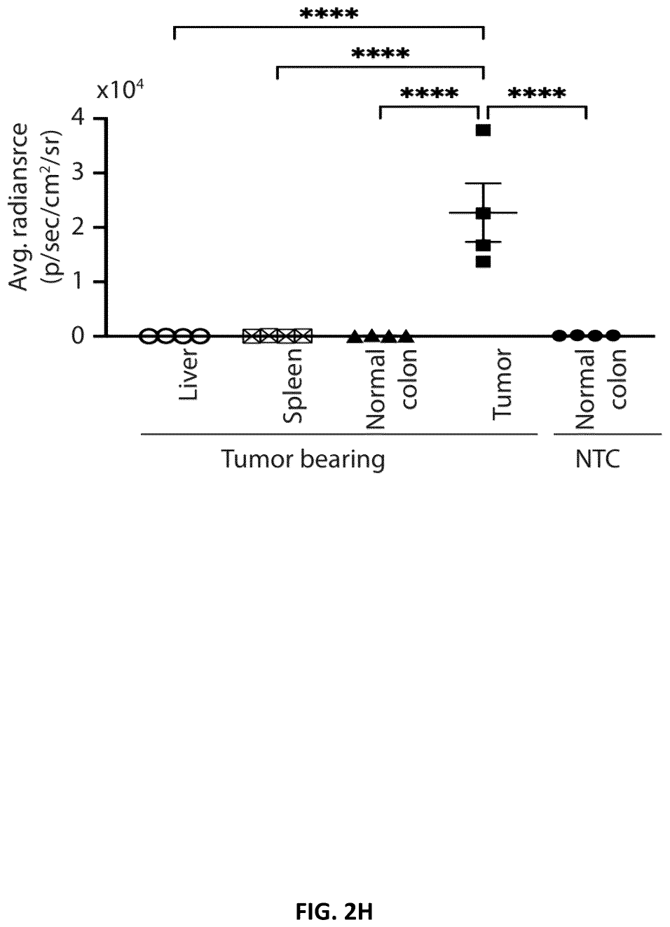

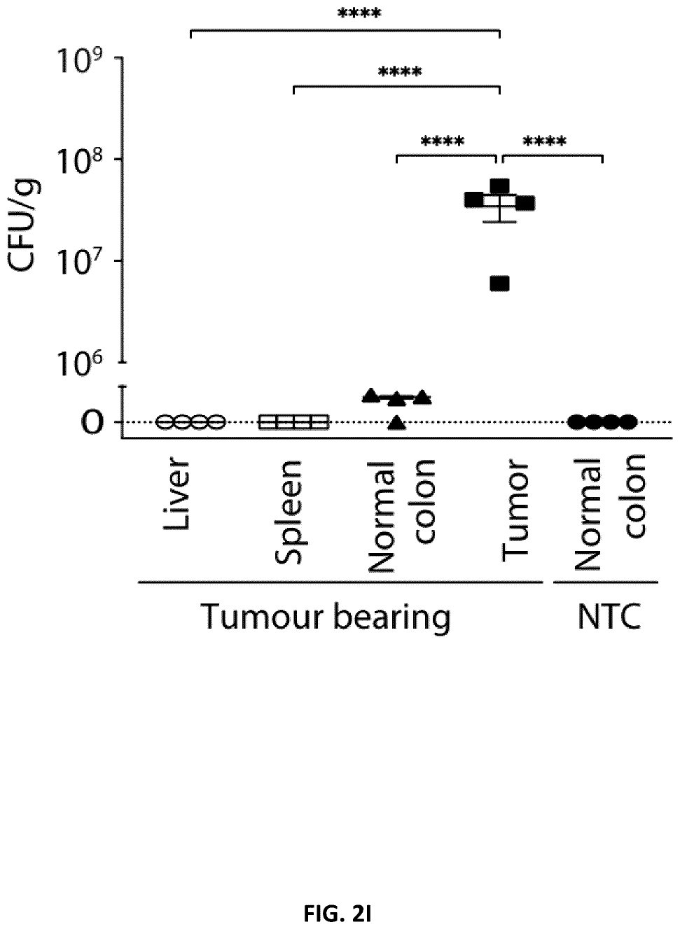

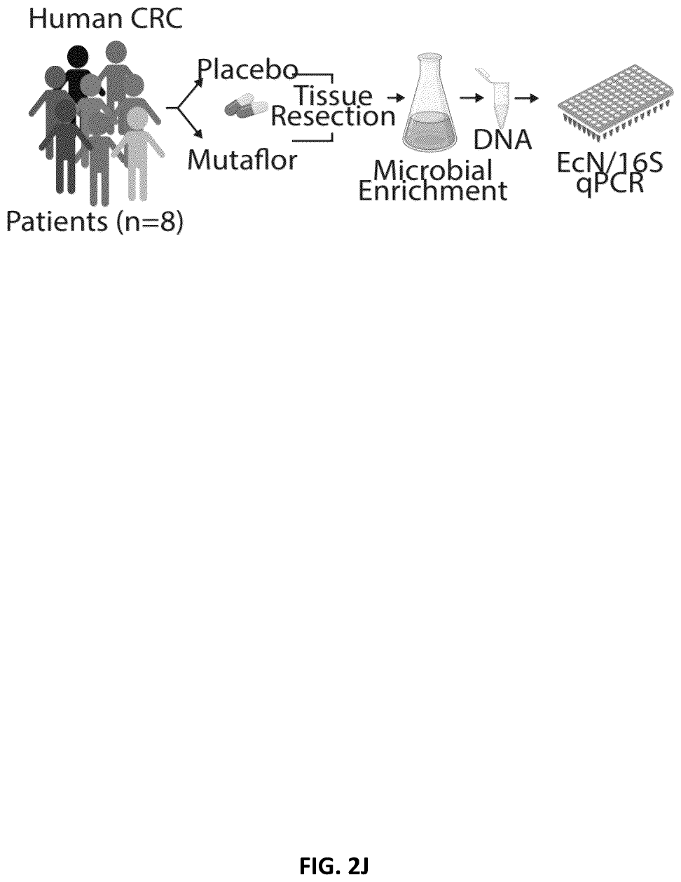

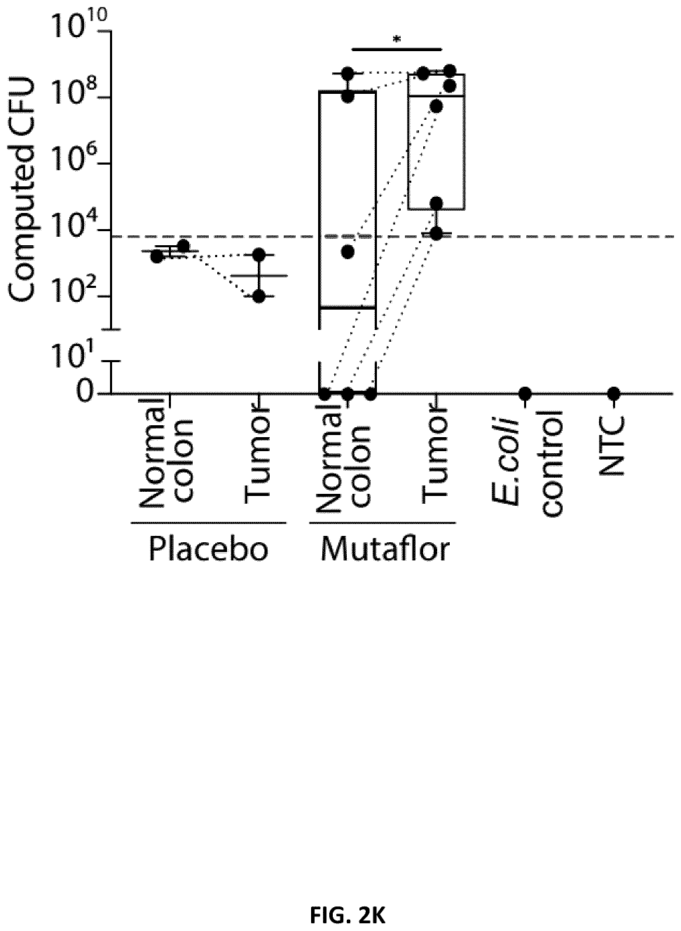

Selective colonization of isolated lesions was investigated by evaluating EcN-lux in two orthotopic models of CRC, representative of MSS and MSI subtypes of disease, whereby murine CRC organoids were injected into the distal colon of recipient mice and tumor grade tracked via weekly colonoscopy (FIG. 2A). To begin, MSS CRC tumor bearing mice were pre-treated with broad-spectrum antibiotics to disrupt the normal microbiota composition, a common phenomenon in gastrointestinal diseases including CRC. EcN-lux was orally delivered and in vivo imaging five days post dosing revealed colocalization of bioluminescent EcN-lux with colon tumors (FIG. 2B-C). Subsequent homogenization and plating of excised organs on antibiotic-selective LB plates confirmed EcN-lux was significantly enriched in tumors compared to adjacent healthy tissue and peripheral organs (FIG. 2D). The median diameter of EcN-lux colonized tumors was 2 mm (+/−1.2 mm), suggesting the size of neoplastic lesions detected using this EcN-lux platform was similar to the size of diminutive (0-5 mm) polyps in humans.

Specific localization of EcN-Lux in these tumors by RNA in situ hybridization, suggested that the bacteria can predominantly be found in nests on the luminal tumor surface and can co-locate with hypoxic tumor regions that may further facilitate bacterial growth within the tumor space (FIG. 2E-F). Similarly, oral EcN-lux dosing of a MSI CRC model (FIG. 2G), without antibiotic pretreatment, resulted in selective, significantly enriched colonization of tumors in comparison to adjacent tissue and organs as measured by ex vivo luminescence imaging and CFU plating (FIG. 2H-I).

### Example 14

### Tumor Colonization of EcN in Human CRC

To explore the translational potential of these pre-clinical findings, a determination was made as to whether gram negative bacteria, such as E. coli, could be visualized in tumor samples from CRC patients using a pan-gram negative lipopolysaccharide (LPS) bacterial stain. Consistent with previous reports of the tumor associated CRC microbiome, LPS bacteria associated with human CRC. A clinical trial was subsequently undertaken to specifically examine EcN colonization in CRC patients. A commercially available, non-genetically modified form of EcN, MUTAFLOR®, or placebo, was orally administered to CRC patients for two weeks, prior to tissue resection. A small number of participants were recruited to this study after early closure due to COVID-19 related trial restrictions and concerns over colibactin producing E coli, such as EcN. Homogenates from matched normal and tumor tissue (n=8 patients) were cultured to enrich for microbial content and DNA was then isolated and subjected to qPCR assays. Despite the smaller than intended number of samples, EcN-specific PCR amplicons indicated significant enrichment of this bacteria in cultures from tumor tissue in patients administered MUTAFLOR®, but not placebo controls (FIG. 2J-K).

### Example 15

### Engineering a Stool and Urine-Based EcN Platform for Non-Invasive Adenoma Tracking

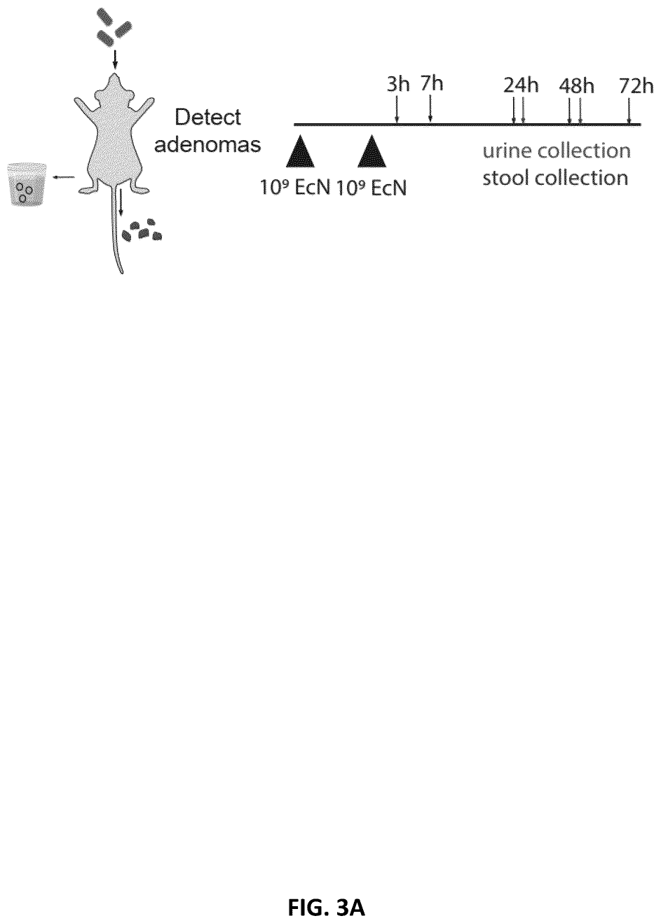

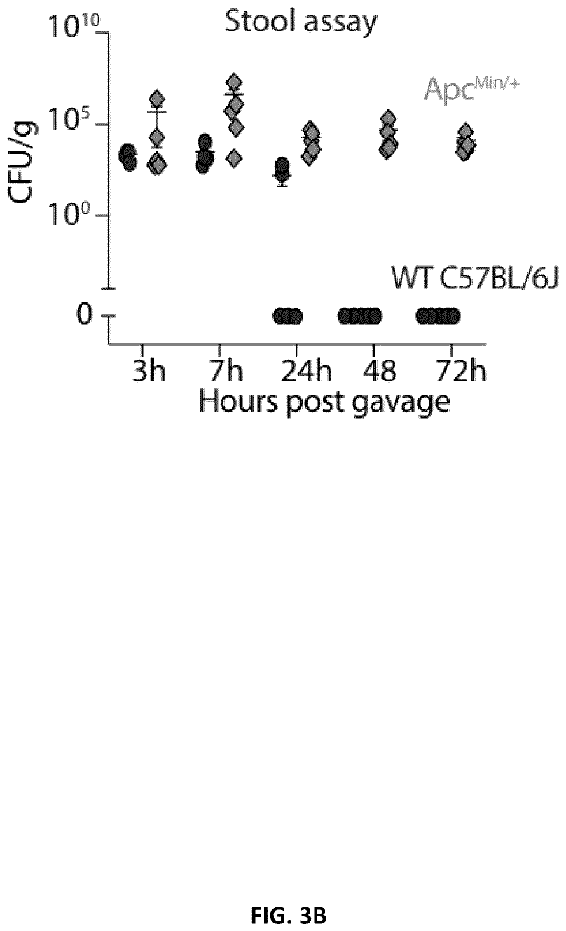

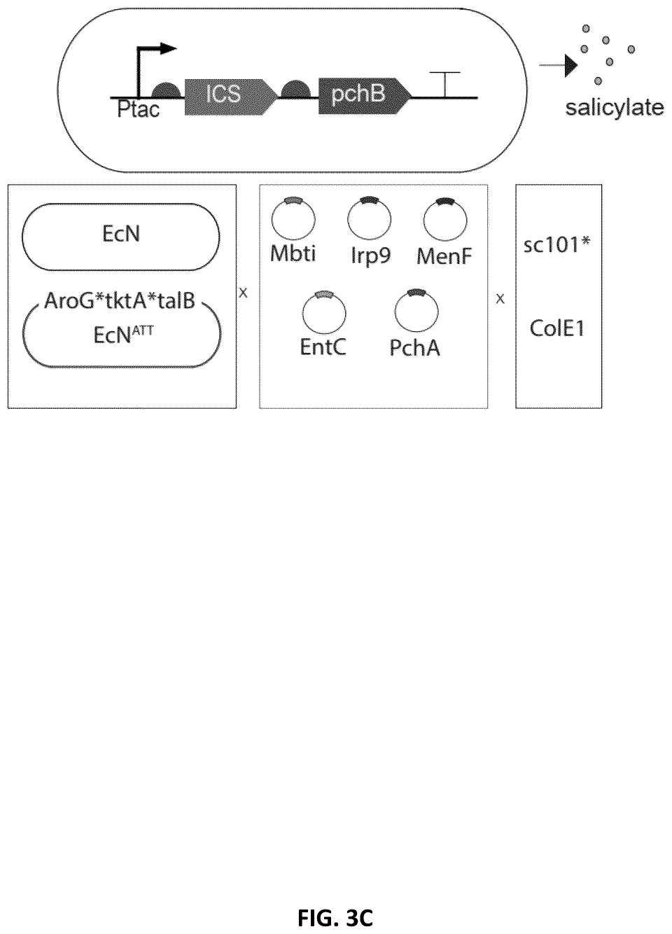

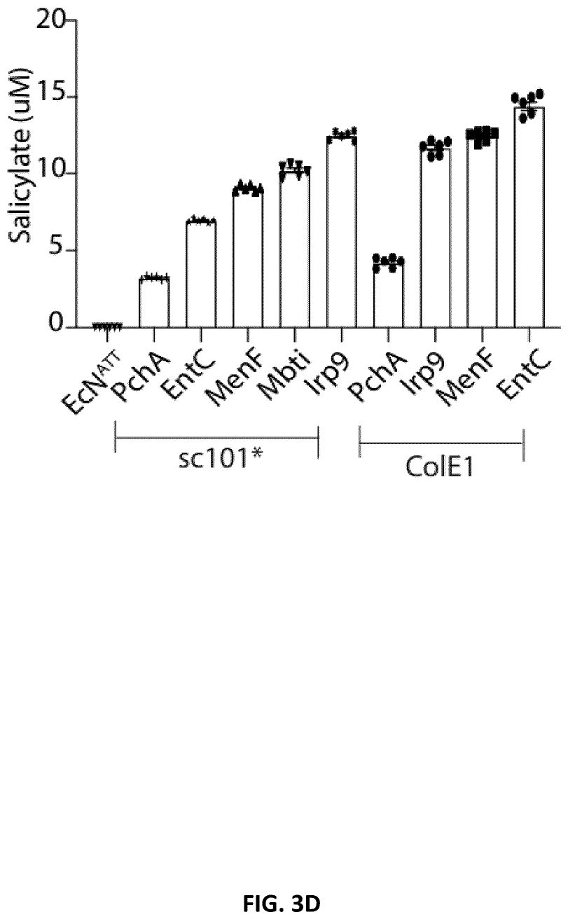

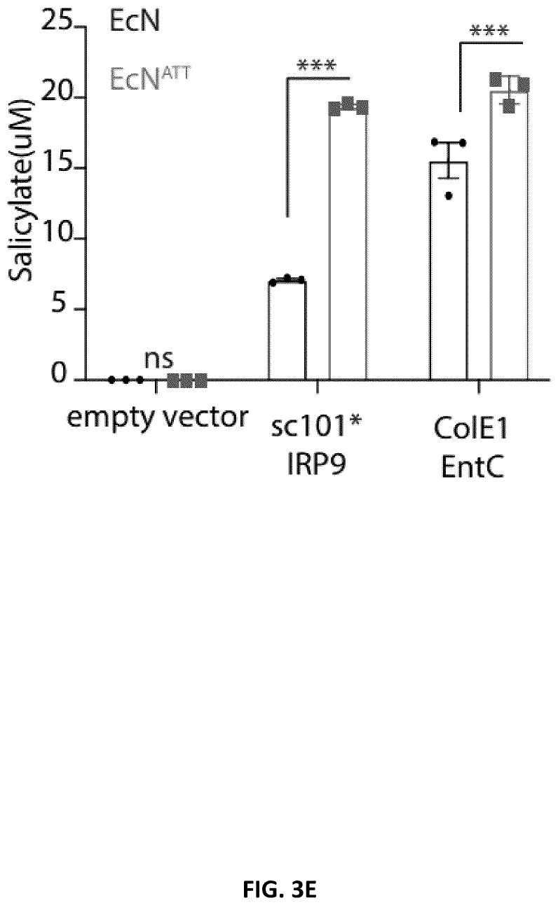

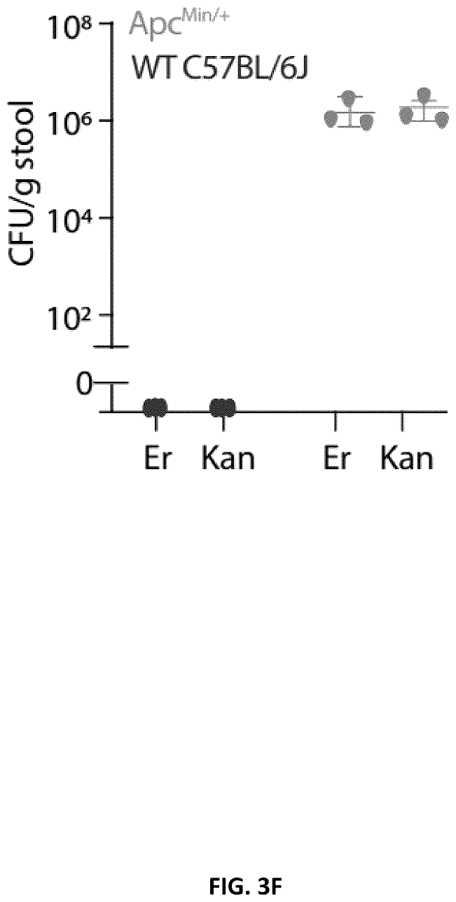

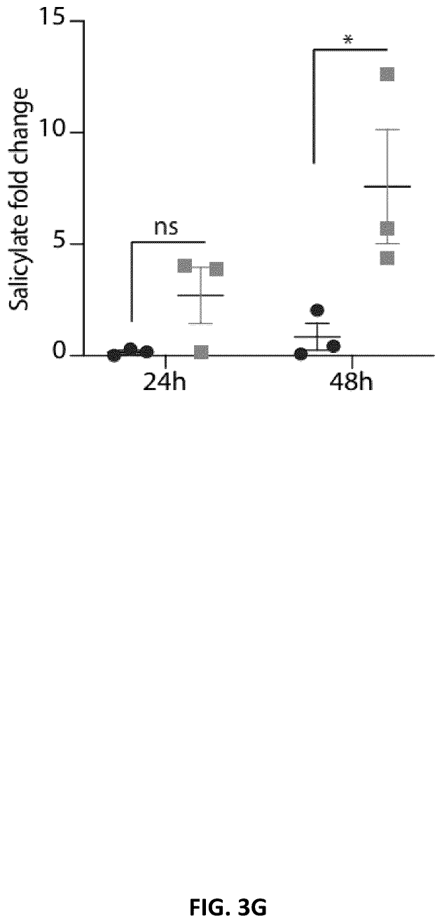

The phenomenon of EcN neoplasia-selective colonization suggested its utility as a platform to monitor adenoma presence. Since stool-based tests are a common non-invasive screening tool available for CRC, we first determined if EcN-lux recoverable in stool could be used to non-invasively monitor the presence of adenomas over time. To do this, we orally dosed both healthy wild-type (WT) and Apc^{Min/+} mice with EcN-lux, collected stool pellets at predetermined time points, homogenized and lastly plated fecal matter on antibiotic-selective LB agar plates (FIG. 3A).

During the first seven hours post-dosing, both WT and Apc^{Min/+} mice had comparable shedding of EcN-lux CFU in their stool, corresponding to material transit time through the gut. However, by twenty-four hours, levels of EcN-lux were undetectable in some healthy mice and by forty-eight hours we were unable to recover EcN-lux from their stool, but were still recoverable in Apc^{Min/+} mice (FIG. 3B). Using the MSI CRC model or the Apc^{Min/+} model dosed with the EcNΔclbA-lux mutant strain, similar retention of EcN-lux in fecal samples in neoplasia-bearing was observed mice compared to normal control mice.

While this stool test could be a useful method for adenoma tracking, a more accessible diagnostic readout is desirable. To do this, EcN were engineered to produce salicylate due to its role in CRC chemoprevention and because it can be feasibly detected in urine. To optimize salicylate production, a library of strain variants were engineered to express genes critical to the shikimate pathway, which is responsible for converting endogenous bacterial chorismite to salicylate. Specifically, genes mbti, irp9, menF, entC, or pchA were cloned onto plasmids also encoding pchB with either low (sc101*) or high (colE1) copy number origins. These plasmids were then cloned into two distinct EcN strains—the EcNΔclbA-lux (EcN) mutant or the EcN^{ATT}ΔclbA-lux (EcN^{ATT}), where the latter included genomically integrated aroG, tkt and talB genes. As these genes are involved in the shikimate and pentose phosphatase pathways, it was hypothesized that their integration would redirect metabolic flux towards increased salicylate production (FIG. 3C). Some variants containing the higher copy plasmid were un-able to grow or contained mutations in the gene of interest, which may be due to the toxic effect of higher levels of salicylate on bacterial cell viability.

Proceeding with the viable EcN^{ATT} strains, liquid-chromatography mass spectrometry (LC-MS) was used to probe for salicylate in supernatant collected from overnight cultures. Generally, higher copy variants produced more salicylate with EcN^{ATT}-EntC-ColE1 producing ˜15 μM salicylate per 10⁹ bacteria (FIG. 3D). Furthermore, comparison of the highest producing strains from both copy number variants, sc101*-Irp9 and EntC-ColE1, demonstrated higher salicylate production when encoded by the EcN^{ATT} mutant compared to the EcN strain (FIG. 3E). Taken together, we determined that the highest producing salicylate variant was the EcN^{ATT}-EntC-ColE1 strain.

To evaluate this optimized strain's diagnostic potential, Apc^{Min/+} and WT mice were dosed with the EcN^{ATT}-EntC-ColE1, and both stool and urine were collected at predetermine timepoints. EcN recovered from stool collected at forty-eight hours post gavage was used to confirm colonization or lack thereof of the strain in Apc^{Min/+} mice and WT. Furthermore, stool was also plated on kanamycin plates selective for the salicylate-encoding plasmid to confirm plasmid retention (FIG. 3F). Urine was collected before dosing to establish a baseline and then again at twenty-four and forty-eight hours after dosing. Salicylate presence was probed for with LC-MS and Apc^{Min/+} mice had up to 5 times more salicylate forty-eight hours after dosing compared to baseline, whereas salicylate levels in our WT mice did not change over time (FIG. 3G). In separate studies, increased salicyluric acid—the primary metabolite of salicylate—was also detected at higher levels in urine. Taken together, these data suggest that engineered bacteria can be delivered to neoplasia-bearing mice, maintain their genetic circuitry, and be used as a proxy for non-invasive adenoma tracking and possible early detection in both fecal matter and urine-based assays.

### Example 16

Treatment with EcN Engineered to Produce Immunotherapeutics Reduces Adenoma Burden and Modifies the Tumor-Immune Microenvironment

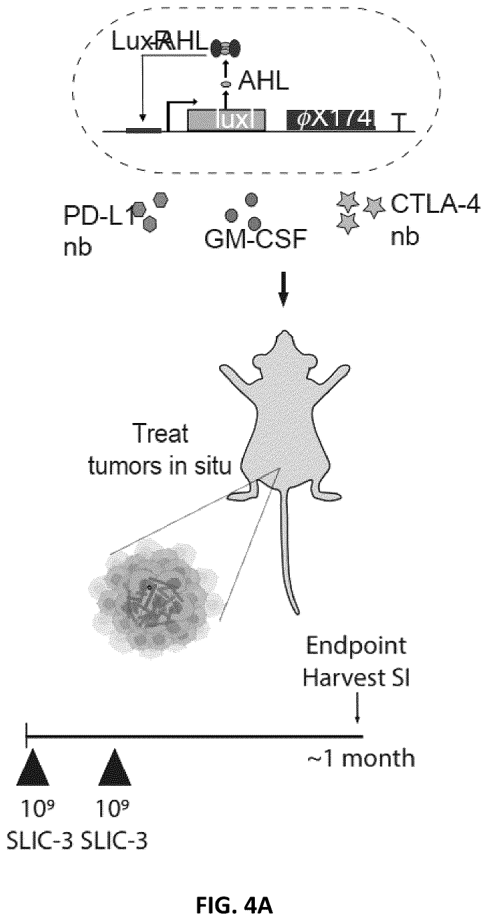

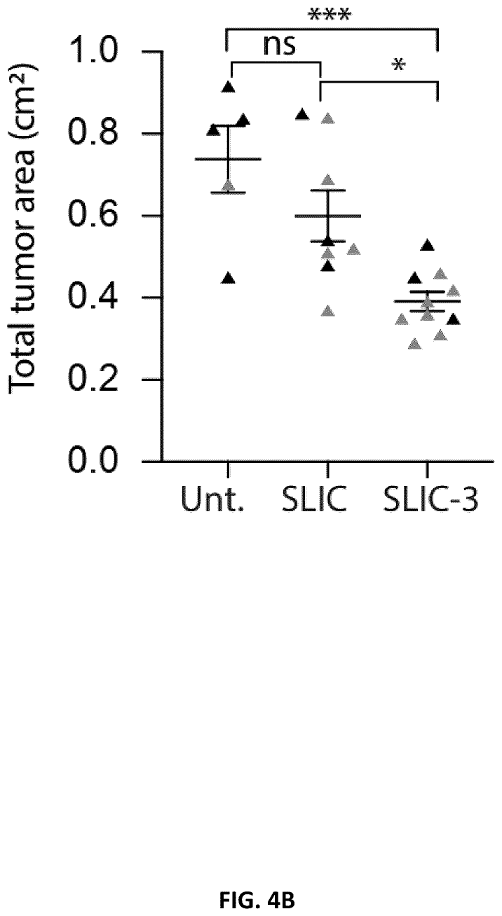

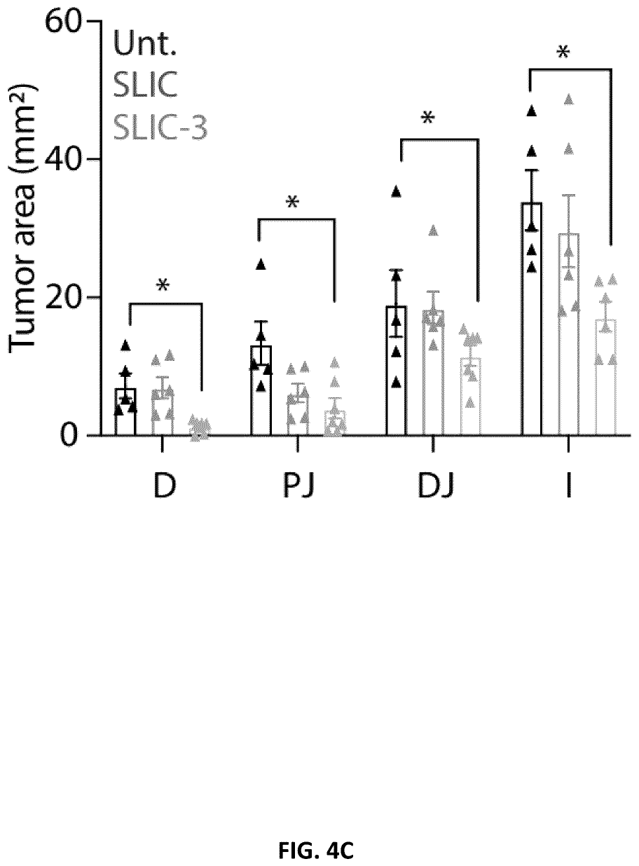

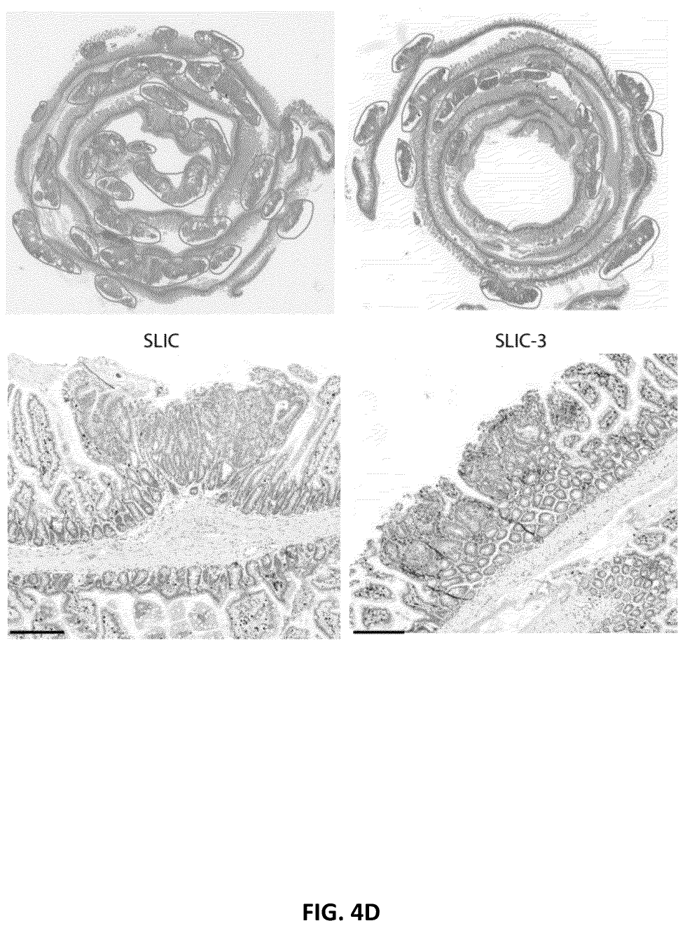

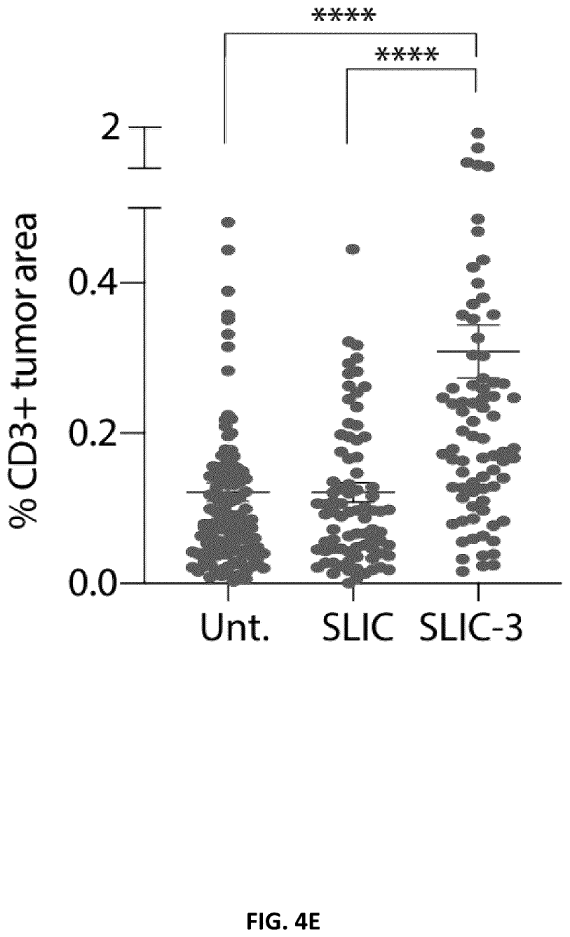

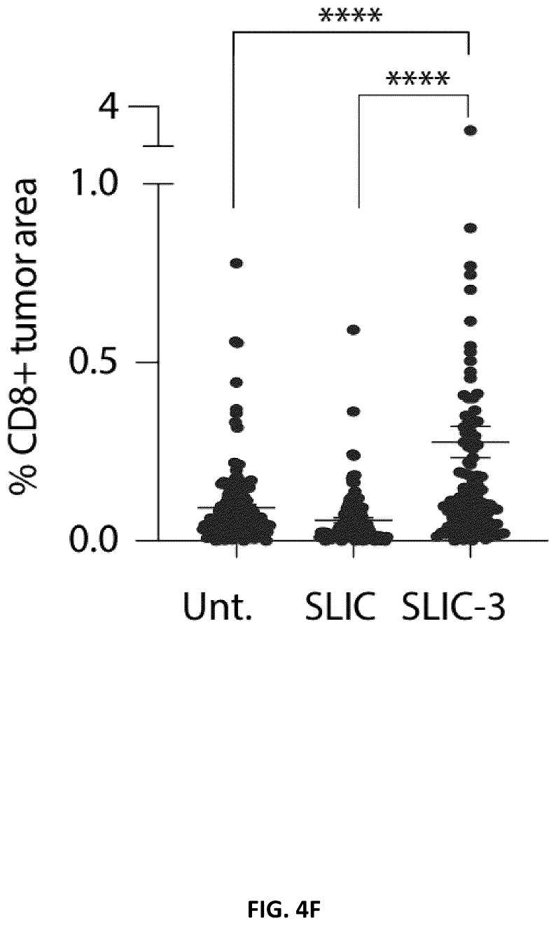

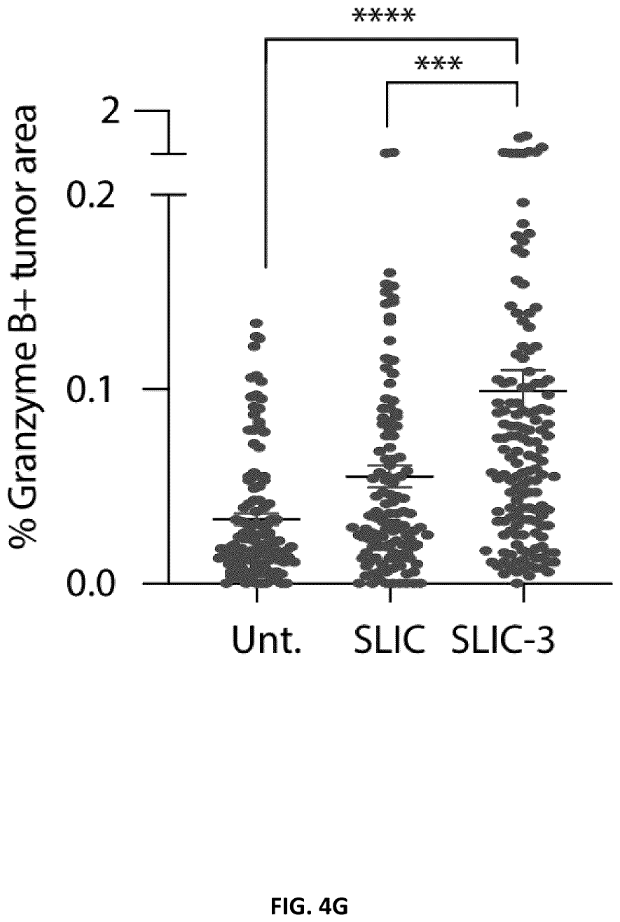

With the ability to non-invasively determine adenoma presence, the screening system was adapted to reduce polyp burden. As the Apc^{Min/+} model is considered to be microsatellite stable (MSS), which traditionally responds poorly to immunotherapeutic approaches, bacteria in this probiotic platform can serve as an immune adjuvant and also a chassis to deliver multiple immunotherapeutic payloads. An EcN-lux strain genomically-encoded with a lysis circuit optimized (SLIC) to maximize immunotherapeutic release was used to deliver nanobodies blocking PD-L1 and CTLA-4 targets and cytokine GM-CSF (SLIC-3) (FIG. 4A). Here, the Apc^{Min/+} mice were either orally dosed twice within 3-4 days with PBS or SLIC-3 and then sacrificed ˜1 month later. Histological analysis of hematoxylin and eosin-stained tumors demonstrate an overall reduction of adenoma area (FIG. 4B) and number by ˜47% with SLIC-3 treatment. Notably, an increased percentage of smaller adenomas was observed in SLIC-3 treated mice, whereas PBS-treated mice tended to have larger adenomas (data not shown). Moreover, this reduction was not specific to a location and was observed throughout the small intestine (FIG. 4C-D). Interrogation of immunophenotype on tissue sections suggested that reduction in adenoma burden is associated with increased infiltration of CD3⁺, CD8⁺ cells and production of Granzyme B within adenomas, suggesting immune-mediated tumor cell killing in SLIC-3 treated mice (FIG. 4E-G). Moreover, there is a trend whereby SLIC alone increases Granzyme B staining when compared to untreated mice, likely due to the inherent immunogenicity of lysed bacteria, underlying the added benefit of leveraging a bacteria-based platform.

While this invention has been disclosed with reference to particular embodiments, it is apparent that other embodiments and variations of the inventions disclosed herein can be devised by others skilled in the art without departing from the true spirit and scope thereof. The appended claims include all such embodiments and equivalent variations.

What is claimed is:

1. A method of detecting the presence of a colorectal tumor in a subject comprising:
(a) administering a programmable bacterial cell to the subject, wherein the programmable bacterial cell comprises:
(i) a first nucleic acid encoding an isochorismate synthase; and
(ii) a second nucleic acid encoding an isochorismate-pyruvate lyase;
(b) obtaining a biological sample comprising salicylate from the subject; and
(c) measuring the amount of salicylate in the biological sample, and detecting the presence of a colorectal tumor in the subject thereby.

2. The method of claim 1, wherein the programmable bacterial cell further comprises a plasmid comprising a third nucleic acid encoding a second isochorismate synthase or a salicylate synthase.

3. The method of claim 1, wherein the programmable bacterial cell belongs to a genus selected from the group consisting of Salmonella, Escherichia, Firmicutes, Bacteroidetes, Lactobacillus, and Bifidobacteria.

4. The method of claim 3, wherein the programmable bacterial cell belongs to the genus Escherichia.

5. The method of claim 4, wherein the programmable bacterial cell is an Escherichia coli Nissle 1917 (EcN) cell.

6. The method of claim 1, wherein the programmable bacterial cell comprises a knockout of the clbA gene (EcNΔclbA).

7. The method of claim 1, wherein the programmable bacterial cell comprises genomically integrated aroG, tkt and talB genes (EcN^{ATT}).

8. The method of claim 1, wherein the programmable bacterial cell comprises a luxCDABE luciferase cassette (EcN-lux).

9. The method of claim 1, wherein the programmable bacterial cell further comprises a synchronized lysis circuit comprising a nucleic acid encoding a quorum-sensing gene, a nucleic acid encoding a lysis gene, a promoter, and a terminator contained on a single operon.

10. The method of claim 9, wherein the programmable bacteria cell further comprises one or more nucleic acids encoding a therapeutic agent selected from the group consisting of:
(a) an antibody that specifically binds to PD-L1;
(b) an antibody that specifically binds to CTLA-4;
(c) a cytokine;
(d) an antibody that specifically binds to PD-L1 and an antibody that specifically binds to CTLA-4;
(e) an antibody that specifically binds to PD-L1 and a cytokine;
(f) an antibody that specifically binds to CTLA-4 and a cytokine; and
(g) an antibody that specifically binds to PD-L1, an antibody that specifically binds to CTLA-4, and a cytokine (e.g., GM-CSF).

11. A method of detecting the presence of a colorectal tumor in a subject comprising:
(a) administering a programmable bacterial cell to the subject, wherein the programmable bacterial cell comprises a knockout of one or more genes encoding a lactate dehydrogenase, a phosphate acetyltransferase, an acetaldehyde-CoA dehydrogenase, and a fumarate reductase, and further comprise:
(i) a first nucleic acid encoding an acetyl-CoA acetyltransferase;
(ii) a second nucleic acid encoding a 3-hydroxybutyryl-CoA dehydrogenase;
(iii) a third nucleic acid encoding a crotonase;
(iv) a fourth nucleic acid encoding a trans-enoyl-CoA reductase; and
(v) a fifth nucleic acid encoding an acyl-CoA thioesterase;
(b) obtaining a biological sample comprising butyrate from the subject; and
(c) measuring the amount of butyrate in the biological sample, and detecting the presence of a colorectal tumor in the subject thereby.

12. The method of claim 11, wherein the programmable bacterial cell comprises a knockout of a gene encoding a lactate dehydrogenase, knockout of a gene encoding a phosphate acetyltransferase, knockout of a gene encoding an acetaldehyde-CoA dehydrogenase, and a knockout of a gene encoding a fumarate reductase.

13. The method of claim 11, wherein the programmable bacterial cell belongs to a genus selected from the group consisting of Salmonella, Escherichia, Firmicutes, Bacteroidetes, Lactobacillus, and Bifidobacteria.

14. The method of claim 13, wherein the programmable bacterial cell belongs to the genus Escherichia.

15. The method of claim 14, wherein the programmable bacterial cell is an Escherichia coli Nissle 1917 (EcN) cell.

16. The method of claim 11, wherein the programmable bacterial cell comprises a knockout of the clbA gene (EcNΔclbA).

17. The method of claim 11, wherein the programmable bacterial cell comprises genomically integrated aroG, tkt and talB genes (EcN^{ATT}).

18. The method of claim 11, wherein the programmable bacterial cell comprises a luxCDABE luciferase cassette (EcN-lux).

19. The method of claim 11, wherein the programmable bacterial cell further comprises a synchronized lysis circuit comprising a nucleic acid encoding a quorum-sensing gene, a nucleic acid encoding a lysis gene, a promoter, and a terminator contained on a single operon.

20. The method of claim 19, wherein the programmable bacteria cell further comprises one or more nucleic acids encoding a therapeutic agent selected from the group consisting of:
(a) an antibody that specifically binds to PD-L1;
(b) an antibody that specifically binds to CTLA-4;
(c) a cytokine;
(d) an antibody that specifically binds to PD-L1 and an antibody that specifically binds to CTLA-4;
(e) an antibody that specifically binds to PD-L1 and a cytokine;
(f) an antibody that specifically binds to CTLA-4 and a cytokine; and
(g) an antibody that specifically binds to PD-L1, an antibody that specifically binds to CTLA-4, and a cytokine (e.g., GM-CSF).


SEQUENCE LISTING (7 sequences)

SEQ ID NO: 1 - DNA, 22 bp, Escherichia coli
  source 1..22: mol_type = other DNA
tcgcgaacgttaaataatcatc

SEQ ID NO: 2 - DNA, 26 bp, Escherichia coli
  source 1..26: mol_type = other DNA
tctgttttagataaggccatgtcttc

SEQ ID NO: 3 - DNA, 23 bp, Escherichia coli
  source 1..23: mol_type = other DNA
acccatcgataccaaatgtatgt

SEQ ID NO: 4 - DNA, 24 bp, Escherichia coli
  source 1..24: mol_type = genomic DNA
tcaatgcgtactcgactattcaaa

SEQ ID NO: 5 - DNA, 16 bp, Escherichia coli
  source 1..16: mol_type = other DNA
cactgacctcaataca

SEQ ID NO: 6 - DNA, 19 bp, Escherichia coli
  source 1..19: mol_type = other DNA
tcctacgggaggcagcagt

SEQ ID NO: 7 - DNA, 26 bp, Escherichia coli
  source 1..26: mol_type = other DNA
ggactaccagggtatctaatcctgtt